(19) United States
(12) Patent Application Publication
(10) Pub. No.: US 2022/0017495 A1
(43) Pub. Date: Jan. 20, 2022

(54) CONJUGATES OF BIVALENT EVANS BLUE DYE DERIVATIVES AND METHODS OF USE

(71) Applicant: THE UNITED STATES OF AMERICA, AS REPRESENTED BY THE SECRETARY, DEPARTMENT OF HEALTH AND HUMAN SERVIC, Bethesda, MD (US)

(72) Inventors: Xiaoyuan CHEN, Bethesda, MD (US); Gang NIU, Bethesda, MD (US); Lixin LANG, Bethesda, MD (US); Rui TIAN, Bethesda, MD (US); Dale O. KIESEWETTER, Bethesda, MD (US)

(21) Appl. No.: 17/294,914

(22) PCT Filed: Jan. 30, 2020
(86) PCT No.: PCT/US2020/015818

§ 371 Date: May 18, 2021

Related U.S. Application Data
(60) Provisional application No. 62/798,763, filed on Jan. 30, 2019.

(51) Int. Cl. C07D 403/12; C07D 403/14; C07D 251/50; C07C 309/50; A61K 51/04; A61K 47/54

(52) U.S. Cl. CPC C07D 403/12; C07D 403/14; C07D 251/50; C07C 309/50; A61K 51/0461; A61K 51/0482; A61K 47/547; A61K 47/545; A61K 51/0497

(57) ABSTRACT

A compound of Formula (I) or a pharmaceutically acceptable ester, amide, solvate, or salt thereof, or a salt of such an ester or amide or a solvate of such an ester amide or salt, is disclosed. Compositions comprising the compound and methods of use are also disclosed. Those dimeric Evans Blue derivatives, denoted as N(tEB)2, reversibly bind two molecules of albumin via the two albumin binding regions of each NtEB in the dimer, resulting in significantly increased binding affinity to albumin and extended circulation half-life in vivo. Further, when the N(tEB)2 is conjugated to a peptide therapeutic, the in situ formation of the complex of N(tEB)2 with two albumin molecules resulted in increased resistance of the peptide therapeutic from proteolysis.

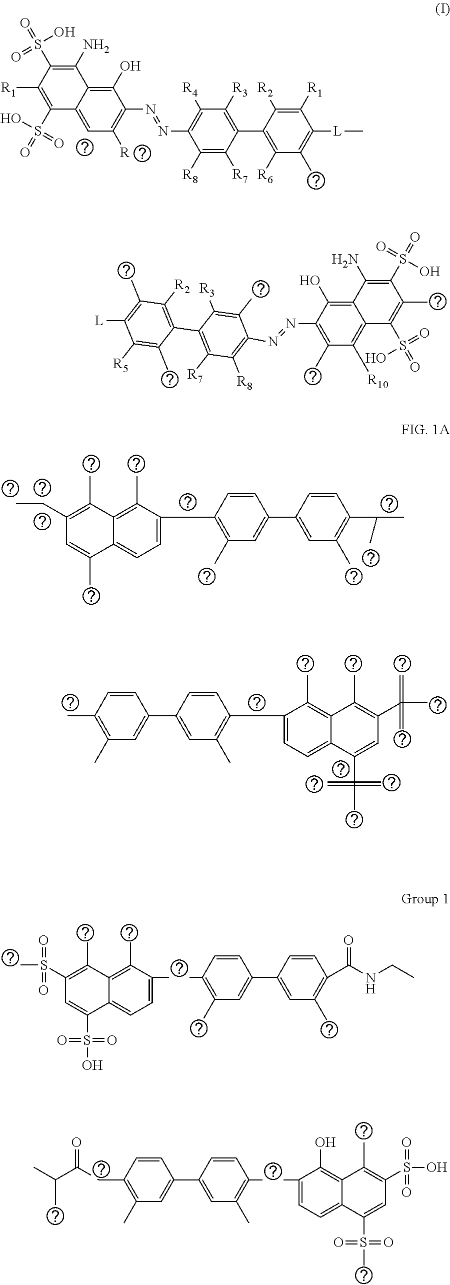

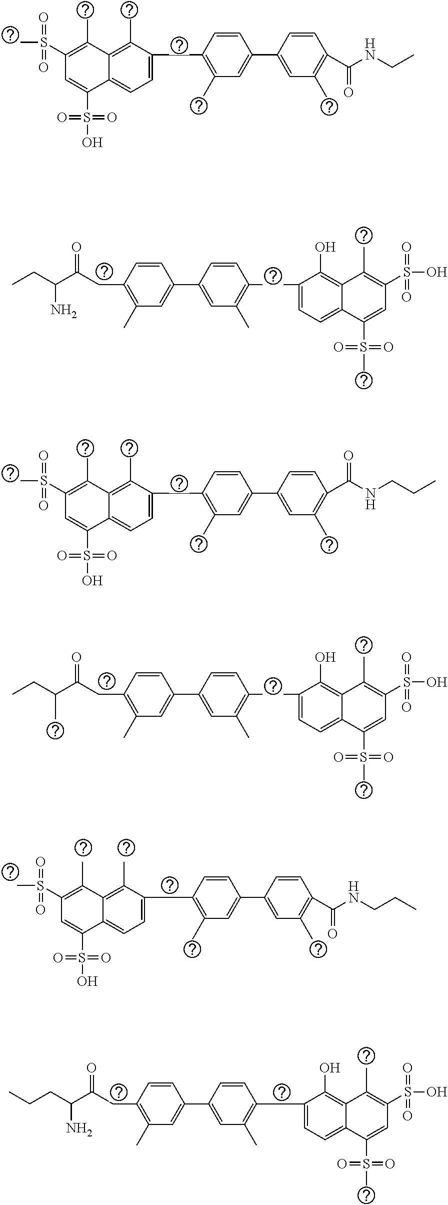

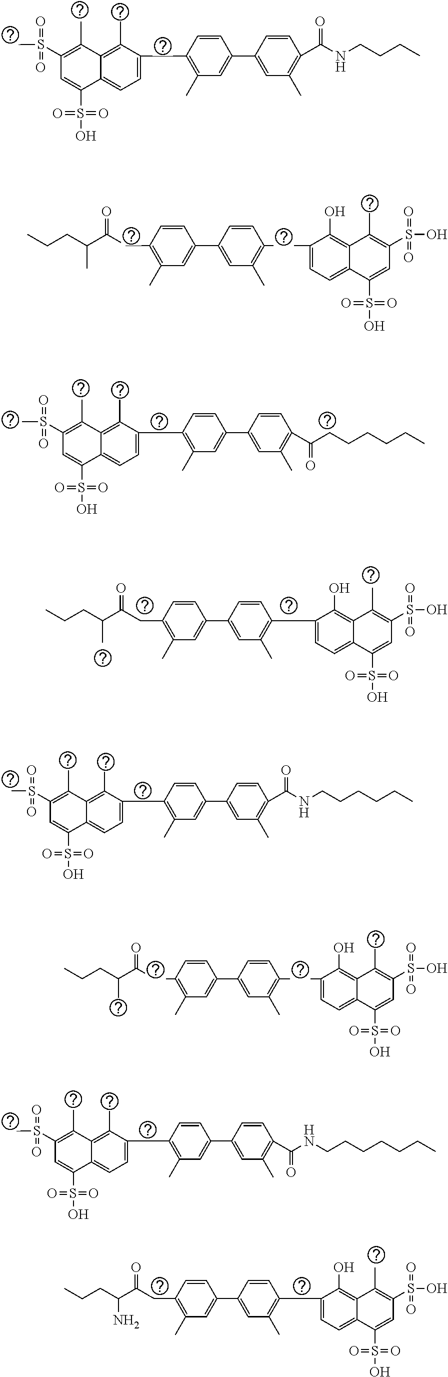

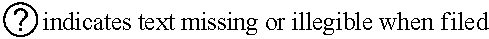

### CROSS-REFERENCE TO RELATED APPLICATIONS

This application claims the benefit of U.S. Provisional Application 62/798,763 filed Jan. 30, 2019, which is incorporated by reference in its entirety.

### STATEMENT OF GOVERNMENT SUPPORT

This invention was made in part with government support from the National Institutes of Health. The government has certain rights in this invention.

### BACKGROUND

The present invention relates to derivatives of Evans Blue dye that are bivalent for binding albumin, and more particularly, to bivalent derivatives of Evans Blue dye that are useful for extending the in vivo half-life of active agents, particularly therapeutic peptides.

The effectiveness of pharmaceuticals depends heavily on pharmacokinetics. In particular, compounds for pharmaceutical use must have sufficient half-life to exert the desired effect on the patient. Various approaches have been used to increase the half-life of pharmaceutical compounds in the body. One method of increasing half-life is to reduce the rate of clearance of the drug from the body, which can be done by inhibition of clearance mechanisms, either through direct modification of the drug, or by addition of other agents which act on the clearance pathways. Reduction of clearance is particularly desired for protein drugs, as they are highly vulnerable to degradation by proteases.

Fusion of protein drugs with large proteins such as albumin or the Fc domain of immunoglobulin G (IgG) can increase drug half-life by increasing the molecular size of the drug and in turn reducing renal clearance. In addition to increasing size, fusion with either albumin or the IgG Fc domain adds functionality to the fused complex and enables interaction with the neonatal Fc receptor (FcRn), which salvages bound ligands from intracellular catabolism by recycling them back to circulation. This interaction with FcRn contributes to the extraordinarily long 21 day serum half-life of albumin and IgG in humans. Therefore, engineering proteins to interact with serum IgG has the potential to significantly increase half-life by reducing both renal clearance and intracellular catabolism. Through these methods the in vivo exposure of the polypeptide or protein therapeutics can be extended. Small molecule drugs may also improve their in vivo pharmacokinetics by association with various plasma components.

Human serum albumin (HSA) has been used as a drug carrier for decades, due to its abundance (35-50 mg/mL) in blood and long systemic circulation. The most popular strategy to “hitchhike” on albumin is to link a drug candidate with an albumin binding moiety (ABM) so that the conjugate binds to circulating albumin in situ. Several FDA-approved drugs incorporate fatty acids as an ABM. However, these fatty acid conjugates tend to have high propensity to accumulate in the liver. The lipophilic nature also increases the difficulty of chemical synthesis and production of these drugs. While other endogenous and exogenous molecules can also bind albumin, a majority of them cannot be used as ABMs because of reduced binding affinity for albumin upon chemical modification. Researchers have used DNA-encoded chemical library and phage display to identify conjugatable ABMs (e.g. 4-(p-iodophenyl) butyric acid derivatives). However, the application of these ABMs has been limited by moderate improvements in the pharmacokinetics of the conjugates or by the mismatch between ABM and drug load. Therefore, an ABM with versatile drug loading ability is still needed to improve drug delivery.

Evans Blue (EB) dye, the tetrasodium salt of 6,6′-{(3,3′-dimethyl[1,1′-biphenyl]-4,4′-diyl)bis[diazene-2,1-diyl]}bis(4-amino-5-hydroxynaphthalene-1,3-disulfonate) (structure shown below), has been an important tool for physiology and pathology, especially for assessing integrity of the blood-brain barrier and vascular permeability, because of its strong affinity for albumin.

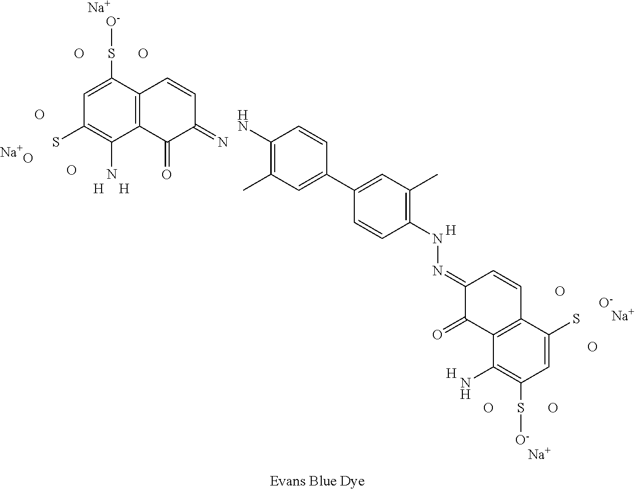

A series of truncated Evans Blue (tEB) derivatives have been developed as ABMs for various applications, including blood pool imaging, tumor vaccination, radioligand therapy, and anti-diabetic treatment (See for example, WO2016/209795, WO2017/196806, International Application No. PCT/US17/054863, and U.S. Application No. 62/633,648.). However, truncation of EB resulted in reduction of its binding affinity for albumin and its fluorescence emission.

Hence, there is a need for a new series of ABMs that can be readily functionalized with imaging/therapeutic molecules, while retaining the high binding affinity and fluorescence efficiency of the parental EB dye.

### SUMMARY

Disclosed herein are compounds of Formula I and methods of use.

In an embodiment, a compound of Formula I or a pharmaceutically acceptable ester, amide, solvate, or salt thereof, or a salt of such an ester or amide or a solvate of such an ester amide or salt,

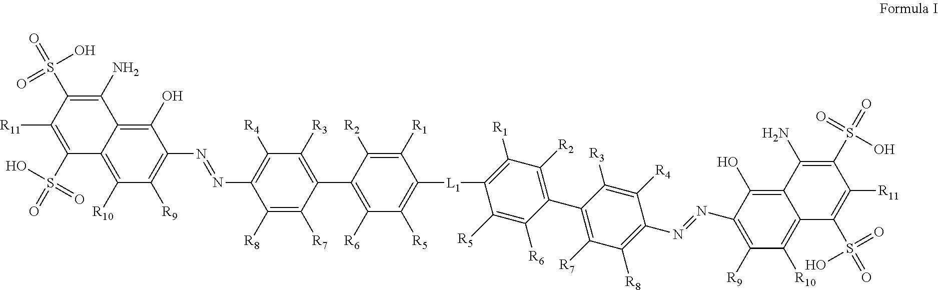

wherein:
R₁, R₂, R₃, R₄, R₅, R₆, R₇, R₈, R₉, R₁₀, and R₁₁ are chosen independently from hydrogen, halogen, hydroxyl, cyano, C₁-C₆alkyl, C₁-C₆alkoxy, C₁-C₆haloalkyl, and C₁-C₆haloalkoxy; and
L₁ is -A₁-B(A₃)-A₂-
wherein
A₁ and A₂ are chosen independently from a bond, —O—, —NH—, —NH(CO)—, —(CO)NH—, —NH(CH₂)ₘ(CO)— wherein m is an integer from 0 to 4, —(CO)(CH₂)ₖNH— wherein k is an integer from 0 to 4, —NH(CS)NH—,

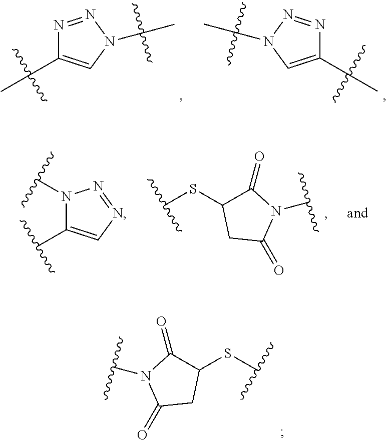 [SMILES: *c1cn(*)nn1 ; *c1cn(*)nn1 ; *c1cnnn1* ; *SC1CC(=O)N(*)C1=O ; *SC1CC(=O)N(*)C1=O]

A₃ is —H, -halogen, —NH₂, —SH, —COOH, or -L₂-R₁₂, wherein
L₂ is —(CH₂)ₚ— wherein p is an integer from 0 to 12, wherein each CH₂ can be individually replaced with —O—, —S—, —NH—, —NH(CO)—, —(CO)NH—, —NH(CS)NH— provided that no two adjacent CH₂ groups are replaced;

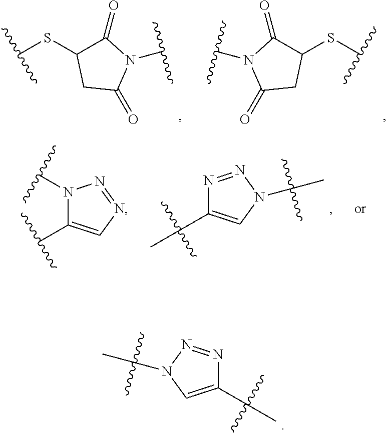 [SMILES: *SC1CC(=O)N(*)C1=O ; *SC1CC(=O)N(*)C1=O ; *c1cnnn1* ; *c1cn(*)nn1 ; *c1cn(*)nn1]

R₁₂ is —H, a chelating group, a crosslinker, or a conjugate; and
B is

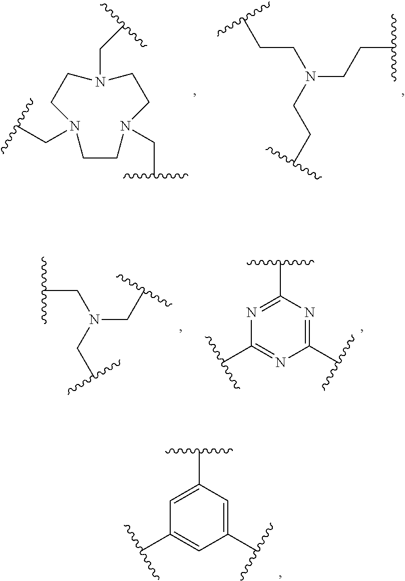 [SMILES: *CN1CCN(C*)CCN(C*)CC1 ; *CCN(CC*)CC* ; *CN(C*)C* ; *c1nc(*)nc(*)n1 ; *c1cc(*)cc(*)c1]

or
—(CH₂)n- wherein n is an integer from 0 to 12, wherein each CH₂ can be individually replaced with —O—, —NH(CO)—, or —(CO)NH— providing no two adjacent CH₂ groups are replaced, and wherein —(CH₂)n- is substituted with one substituent A₃.

Compositions comprising a compound of Formula I and a carrier are also disclosed.

A method of treating or diagnosing diabetes in a mammal is disclosed. In an embodiment the method comprises administering to the mammal a compound of Formula I, optionally in combination with one or more additional active ingredients.

A method of increasing the in vivo half-life of target molecule is disclosed. In an embodiment, the method comprises covalently coupling the compound of Formula I to a target molecule.

A method of in vivo imaging is disclosed. In an embodiment, the method comprises administering to a subject a compound of Formula I.

The above described and other features are exemplified by the following figures and detailed description.

### BRIEF DESCRIPTION OF THE DRAWINGS

The following figures are exemplary embodiments.

FIGS. 1A-E show the structures of the virtual library of tEB dimers ((tEB)₂) with different linkers screened by computational modeling. In the general structure for dimerizing tEB shown at the top of FIG. 1A, the spacer R between the tEB monomers is tunable with respect to length, linkage moiety to each of the tEB monomers (e.g., a thiourea, a peptide bond,

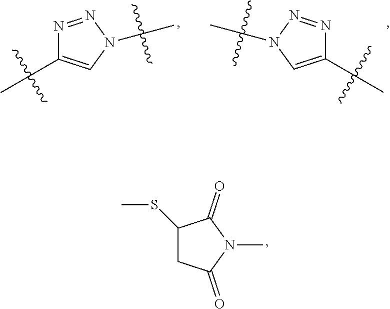

and the like), and functional moiety R′. Exemplary R′ in the screened library include hydrogen and NOTA. FIG. 1E shows tEB dimer structures in which the spacer R is designed to include a NOTA group in the backbone.

FIG. 2 shows the model structure resulting from docking Nt(EB)2 with two human serum albumin (HSA, DIB: 1E78) molecules.

FIG. 3 shows (c) Front projection of the most preferable binding structure for N(tEB)₂ and HSA determined through simulated docking poses for N(tEB)₂ and HSA. (d) Side projection of the binding pose for N(tEB)₂ and HSA, showing one inserted and bound albumin binding moiety head of the N(tEB)₂ and the other head free and available for binding to a second albumin. (e) The detailed docking poses and interaction for N(tEB)₂ and HSA.

FIGS. 4A-4C show the synthetic scheme for N(tEB)₂ 1.

FIG. 5 shows the synthetic scheme for maleimide-derivatized N(tEB)₂ 2

FIGS. 6A-6C shows characterization of the interaction between N(tEB)₂ and albumin by Atomic Force Microscope (AFM). A) AFM images showing in vitro N(tEB)₂-albumin dimers. B) AFM images showing in vitro NtEB-albumin monomers. C) Quantification of mixture of the N(tEB)₂ and NtEB with HSA by molar ratio of 1:1, 10:1 and 1:10, respectively.

FIG. 6D presents graphs of the Dynamic Light Scattering (DLS) analysis showing the diameter of N(tEB)₂ and NtEB complex when mixed with albumin.

FIG. 6E presents fluorescence spectra comparing the fluorescence intensity of EB, NEB and N(tEB)₂ with equivalent molar concentration in both PBS or HSA solution.

FIG. 6F is a plot comparing the relative quantum yield (QY) and relative quantum efficiency (QE) of N(tEB)₂ and NtEB (both of QY and QE of NtEB were artificially set as “1”).

FIG. 7 presents kinetic PET images taken of healthy mice at various time points after injection with ¹⁸F-labeled N(tEB)₂ or ¹⁸F-labeled NtEB, respectively (a) and time-activity plots determined over the heart from the PET images to obtain the half-life of the ¹⁸F-labeled N(tEB)₂ and ¹⁸F-labeled NtEB by two-phase linear regression of the data.

FIG. 8 shows quantification of tumor uptake of ⁶⁴Cu labeled N(tEB)₂ and NtEB in A) U-87MG, B) UM-22B and C) INS-1 tumor mouse xenografts at 1, 4, 24, and 48 h p.i., respectively. Panel D) is a graph showing time-activity curves (TAC) of ROIs over heart regions with ⁶⁴Cu labeled N(tEB)₂ or NtEB.

FIG. 9 shows biodistribution of ⁶⁴Cu labeled N(tEB)₂ and NtEB in a) U-87MG, b) INS-1, and c) UM-22B xenografts at 48 h p.i.

FIG. 10 compares tumor retention of ⁶⁴Cu labeled mouse IgG, NtEB, and N(tEB)₂: A) PET images at 48 hours post-injection; B) quantification of tumor uptake in U-87MG tumor xenografts at different time points post-injection; C) graph of time activity curves (TAC) over heart post-injection.

FIG. 11 shows in vivo lymphatic imaging with N(tEB)₂-albumin dimer, NtEB-albumin, and EB-albumin of the sentinel lymph nodes (LN) and migration process within lymphatic vessels. A) The scheme of lymphatic mapping for comparing fluorescence brightness and migration speed between the three compounds. B) Comparison of the fluorescence lymphatic imaging of N(tEB)₂ (left hind limb) with NtEB (right hind limb) at 60 min and 120 min p.i., respectively (upper 4 panels), and N(tEB)₂ (left hind limb) with EB (right hind limb) at the same time points, respectively (bottom 4 panels). C) Comparison of N(tEB)₂, NtEB and EB dye for fluorescence imaging of the popliteal and sciatic LNs at different time points.

FIG. 12 shows in vivo lymphatic PET imaging with N(tEB)₂-albumin dimer, NtEB-albumin, and EB-albumin. A) The scheme of lymphatic PET imaging. B) ¹⁸F labeled N(tEB)₂ and NtEB for popliteal and sciatic LNs PET imaging at different time points. C) Time activity curves (TAC) of both popliteal and sciatic LNs using ¹⁸F labeled N(tEB)₂ and NtEB, respectively. D) Comparison of the time interval between popliteal LNs and sciatic LNs detection using ¹⁸F labeled N(tEB)₂ and NtEB, respectively.

FIG. 13 presents graphs of A) kinetics of tryptic degradation of exendin-4 as free exendin-4, N(tEB)₂-exendine-4-albumin, or NtEB-exendin-albumin; and B) kinetics of appearance of a tryptic fragment of exendin-4 for free exendin-4, N(tEB)₂-exendine-4-albumin, or NtEB-exendin-albumin.

FIG. 14 presents data showing therapeutic efficacy of N(tEB)₂-exendin-4 in type 2 diabetes mellitus (T2DM) mice. a) Plasma concentration of exendin-4 after administering an equivalent dose of exendin-4 in both the NtEB-exendin-4 and N(tEB)₂-exendin-4 compounds as well as free exendin-4 (n=3 per group). Error bars represent the mean±st.d. of three biological replicates. b and c) Long-term blood glucose monitoring in T2DM after treatment of exendin-4, NtEB-exendin-4, N(tEB)₂-exendin-4, and semaglutide, respectively. d) The time window from the 50% reduction of the glucose level to rebound to the original level. Error bars represent the mean±st.d. of three replicates.

### DETAILED DESCRIPTION

Disclosed herein are dimeric Evans Blue derivatives, denoted as N(tEB)₂, which are bivalent for albumin binding. The N(tEB)₂ reversibly bind two molecules of albumin via the two albumin binding regions of each NtEB in the dimer, resulting in significantly increased binding affinity to albumin and extended circulation half-life in vivo. Further, when the N(tEB)₂ is conjugated to a peptide therapeutic, the in situ formation of the complex of N(tEB)₂ with two albumin molecules resulted in increased resistance of the peptide therapeutic from proteolysis.

### Terminology

Compounds are described using standard nomenclature. Unless defined otherwise, all technical and scientific terms used herein have the same meaning as is commonly understood by one of skill in the art to which this invention belongs.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely for illustration and does not pose a limitation on the scope of the invention unless otherwise claimed. No language in the specification should be construed as indicating any non-claimed element as essential to the practice of the invention.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe. Thus compositions slightly outside the cited ranges are also encompassed by the scope of the present claims.

An “active agent” is any compound, element, or mixture that when administered to a patient alone or in combination with another agent confers, directly or indirectly, a physiological effect on the patient.

The terms “comprising,” “including,” and “containing” are non-limiting. Other non-recited elements may be present in embodiments claimed by these transitional phrases. Where “comprising,” “containing,” or “including” are used as transitional phrases other elements may be included and still form an embodiment within the scope of the claim. The open-ended transitional phrase “comprising” encompasses the intermediate transitional phrase “consisting essentially of” and the close-ended phrase “consisting of.”

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“—”) that is not between two letters or symbols is used to indicate a point of attachment for a substituent.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g., C₁-C₈alkyl, C₁-C₄alkyl, and C₁-C₂alkyl. When C₀-Cₙ alkyl is used herein in conjunction with another group, for example, —C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, iso-propyl, n-butyl, 3-methylbutyl, t-butyl, n-pentyl, and sec-pentyl.

“Alkenyl” is a branched or straight chain aliphatic hydrocarbon group having one or more carbon-carbon double bonds that may occur at any stable point along the chain, having the specified number of carbon atoms. Examples of alkenyl include, but are not limited to, ethenyl and propenyl.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, iso-propoxy, n-butoxy, 2-butoxy, t-butoxy, n-pentoxy, 2-pentoxy, 3-pentoxy, iso-pentoxy, neo-pentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound to the group it substitutes by a sulfur bridge (—S—). Similarly, “alkenyloxy”, “alkynyloxy”, and “cycloalkyloxy” refer to alkenyl, alkynyl, and cycloalkyl groups, in each instance covalently bound to the group it substitutes by an oxygen bridge (—O—).

“Halo” or “halogen” means fluoro, chloro, bromo, or iodo, and are defined herein to include all isotopes of same, including heavy isotopes and radioactive isotopes. Examples of useful halo isotopes include ¹⁸F, ⁷⁶Br, and ¹³¹I. Additional isotopes will be readily appreciated by one of skill in the art.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

Unless substituents are otherwise specifically indicated, each of the foregoing groups can be unsubstituted or substituted, provided that the substitution does not significantly adversely affect synthesis, stability, or use of the compound. “Substituted” means that the compound, group, or atom is substituted with at least one (e.g., 1, 2, 3, or 4) substituents instead of hydrogen, where each substituent is independently nitro (—NO2), cyano (—CN), hydroxy (—OH), halogen, thiol (—SH), thiocyano (—SCN), C1-6 alkyl, C2-6 alkenyl, C2-6 alkynyl, C1-6 haloalkyl, C1-9 alkoxy, C1-6 haloalkoxy, C3-12 cycloalkyl, C5-18 cycloalkenyl, C6-12 aryl, C7-13 arylalkylene (e.g., benzyl), C7-12 alkylarylene (e.g, toluyl), C4-12 heterocycloalkyl, C3-12 heteroaryl, C1-6 alkyl sulfonyl (—S(═O)2-alkyl), C6-12 arylsulfonyl (—S(═O)2-aryl), or tosyl (CH3C6H4SO2-), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the compound or group, including those of any substituents.

“The terms “polypeptide”, “peptide”, and “protein” are used interchangeably herein to refer to a molecule formed from the linking, in a defined order, of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms also apply to amino acid polymers, or “peptidomimetics”, in which one or more amino acid residue is an artificial chemical mimetic of a corresponding naturally occurring amino acid, as well as to naturally occurring amino acid polymers and non-naturally occurring amino acid polymer.

“Pharmaceutical compositions” means compositions comprising at least one active agent, such as a compound or salt of Formula I, and at least one other substance, such as a carrier. Pharmaceutical compositions meet the U.S. Food and Drug Administration's good manufacturing practice (GMP) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

A “target molecule” is a molecule having a desired activity. The molecules can be small molecules, peptides, proteins, nucleic acids, or other kinds of molecules. Examples of a target molecule include an active agent, a marker compound, a fluorescent tag, a pharmaceutically active agent, a toxin, a diagnostic agent, a radioactive agent, a contrast agent, an imaging agent, a nanoparticle, a quantum dot, a liposome, a liposome precursor, a micelle, an antibody, a protein, a peptide, a peptidomimetic, a nucleic acid, a nucleic acid complex, a cytokine, and a hormone.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A significant change is any detectable change that is statistically significant in a standard parametric test of statistical significance such as Student's T-test, where p<0.05.

Chemical Description

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

Formula I includes all pharmaceutically acceptable salts of Formula I.

Compounds of Formula I may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

A “chelating group” or “chelator” is a ligand group which can form two or more separate coordinate bonds to a single central atom, which is usually a metal ion. Chelating groups as disclosed herein are organic groups which possess multiple N, O, or S heteroatoms, and have a structure which allows two or more of the heteroatoms to form bonds to the same metal ion.

A “crosslinking group” or “crosslinker” is a functional group which has a reactive moiety that can chemically react with a specific functional group (e.g., a primary amine, a sulfhydryl, etc.) on a target molecule, for example a peptide, to covalently join the crosslinker and target molecule.

A “conjugate” is a product of reaction between a crosslinker and a target molecule.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, iso-propanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

### EMBODIMENTS

Disclosed herein are compounds that are derivatives or conjugated derivatives of a truncated Evans Blue dye dimer having the compound of Formula I illustrated below, or a pharmaceutically acceptable ester, amide, solvate, or salt thereof, or a salt of such an ester or amide or a solvate of such an ester amide or salt:

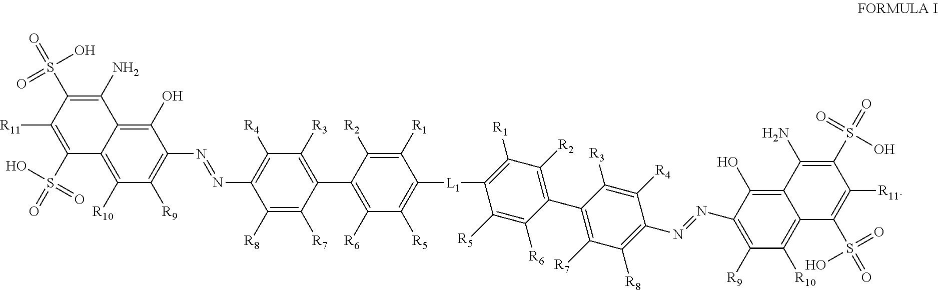

In Formula I, the substituents R₁, R₂, R₃, R₄, R₅, R₆, R₇, R₈, R₉, R₁₀, and R₁₁ are chosen independently from hydrogen, halogen, hydroxyl, cyano, C₁-C₆alkyl, C₁-C₆alkoxy, C₁-C₆haloalkyl, and C₁-C₆haloalkoxy. In an embodiment, R₁ and R₄ are chosen independently from halogen, hydroxyl, cyano, C₁-C₆alkyl, C₁-C₆alkoxy, C₁-C₆haloalkyl, and C₁-C₆haloalkoxy, preferably R₁ and R₄ are chosen independently from C₁-C₆alkyl.

In an embodiment, R₁ and R₄ are each methyl, and R₂, R₃, R₅, R₆, R₇, R₈, R₉, R₁₀, and R₁₁ are each hydrogen.

The linking group L₁ has the structure -A₁-B(A₃)-A₂-. A₁ and A₂ are chosen independently from a bond, —O—, —NH—, —NH(CO)—, —(CO)NH—, —NH(CH₂)ₘ(CO)— wherein m is an integer from 0 to 4, —(CO)(CH₂)ₖNH— wherein k is an integer from 0 to 4, —NH(CS)NH—,

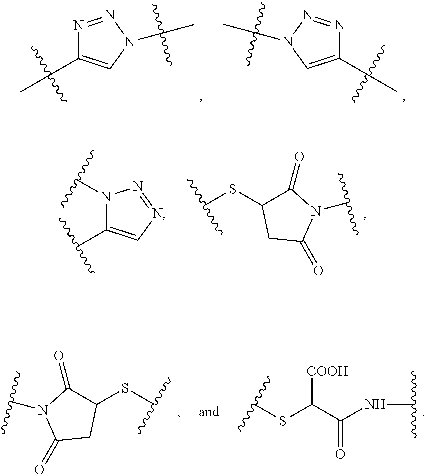 [SMILES: *c1cn(*)nn1 ; *c1cn(*)nn1 ; *c1cnnn1* ; *SC1CC(=O)N(*)C1=O ; *SC1CC(=O)N(*)C1=O ; *NC(=O)C(S*)C(=O)O]

A₃ is —H, -halogen, —NH₂, —SH, —COOH, or -L₂-R₁₂. The linking group -L₂- is one of —(CH₂)ₚ— wherein p is an integer from 0 to 12, wherein each CH₂ can be individually replaced with —O—, —S—, —NH—, —NH(CO)—, —(CO)NH—, —NH(CS)NH— provided that no two adjacent CH₂ groups are replaced;

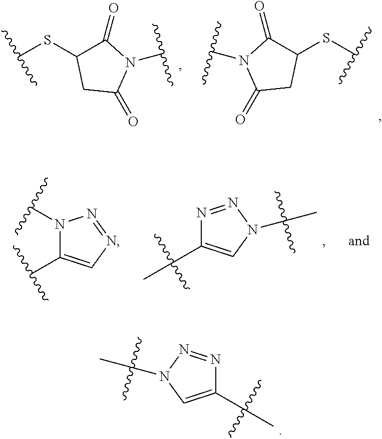 [SMILES: *SC1CC(=O)N(*)C1=O ; *SC1CC(=O)N(*)C1=O ; *c1cnnn1* ; *c1cn(*)nn1 ; *c1cn(*)nn1]

The group —R₁₂ is —H, a chelating group, a crosslinker, or a conjugate.

R₁₂ may be a chelating group. The chelating group can be a macrocyclic moiety, such as a 1,4,7-triazacyclononane-N,N′,N″-triacetic acid (NOTA) group, a 1,4,7,10-Tetraazacyclododecane-1,4,7,10-tetraacetic acid (DOTA) group, mercaptoacetyltriglycine (MAG₃), dipicolylamine ethanoic acid (DPA), cyclodextrin, crown ether, or porphyrin, or may be a linear moiety such as a 1,4,7-triazaheptane-1,4,7,7-tetracetic acid group (DTPA), but is not limited thereto. Chemical structures of these and some other chelating compounds and groups are shown below.

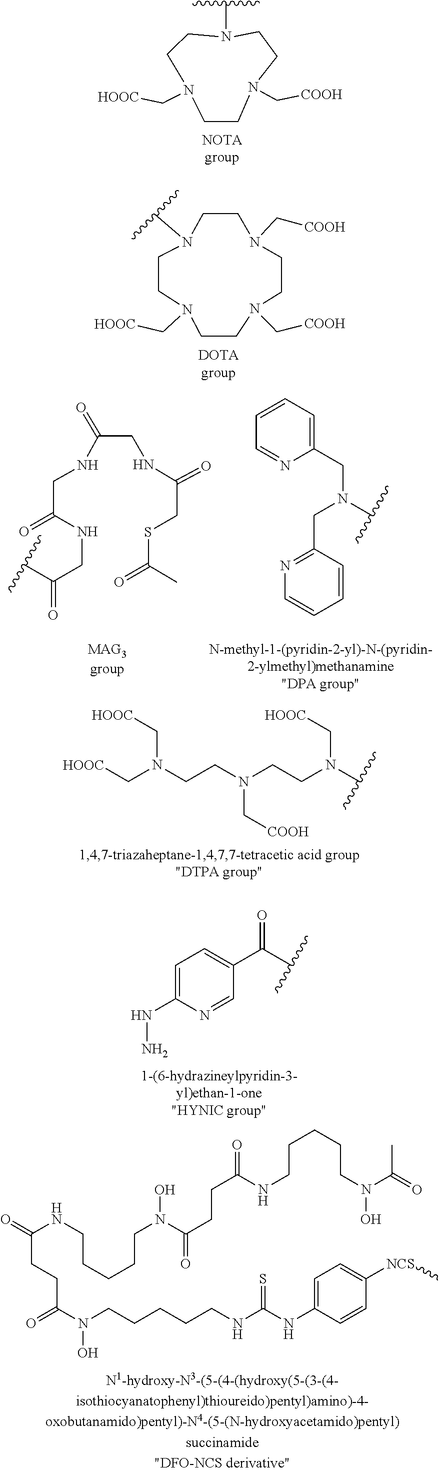

In certain embodiments, R₁₂ is preferably

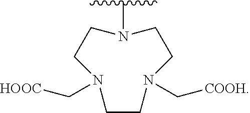 [SMILES: *N1CCN(CC(=O)O)CCN(CC(=O)O)CC1]

Many crosslinkers are known in the art, and available commercially, for conjugating two molecules. Examples of a crosslinker include

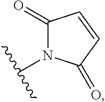 [SMILES: *N1C(=O)C=CC1=O]

### —SH,

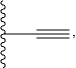 [SMILES: *C#C]

### and —N₃.

Examples of a conjugate include

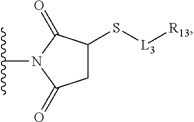

### —SR₁₃,

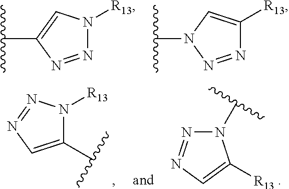

In the conjugates, the group R₁₃ is a target molecule that has reacted with a crosslinking moiety in such a way that its activity is not significantly changed. R₁₃ can be hydrogen, a marker compound, a fluorescent tag, a pharmaceutically active agent, a toxin, a radioactive agent, a contrast agent, a nanoparticle, a quantum dot, a liposome, a liposome precursor, a micelle, an antiangiogenic compound, an antibody, a protein, a peptide, a peptidomimetic, a nucleic acid, a nucleic acid complex, or a cytokine. Preferably R₁₃ is a marker compound, a fluorescent tag, a pharmaceutically active agent, a toxin, a radioactive agent, a contrast agent, an antibody, a protein, a peptide, a peptidomimetic, a nucleic acid, a nucleic acid complex, or a cytokine. More preferably R₁₃ is a peptide. L₃ is —(CH₂)_{q}— wherein q is an integer from 0 to 12, and each CH₂ can be individually replaced with —O—, —NH(CO)—, or —(CO)—NH—, providing no two adjacent CH₂ groups are replaced.

The pharmaceutically active agent may include any therapeutic class of compound, for example an anti-diabetic agent, an anti-cancer agent, an anti-biotic agent, an anti-thrombotic agent (e.g., an anti-coagulant, an anti-platelet, a thrombolytic agent, etc.), a hormone, a cytokine, or an analog thereof. Examples of suitable active agents include insulin, an insulin analog, IL-2, IL-5, GLP-1, BNP, IL 1-RA, KGF, ancestim, GH, G-CSF, CTLA-4, myostatin, Factor VII, Factor VIII, Factor IX, Exendin-4, exendin (9-39), octreotide, bombesin, RGD peptide (arginylglycylaspartic acid), vascular endothelial growth factor (VEGF), interferon (IFN), tumor necrosis factor (TNF), asparaginase, adenosine deaminase, a therapeutic fragment of any of the foregoing, a derivative of any of the foregoing, calicheamycin, auristatin, doxorubicin, maytansinoid, taxane, ecteinascidin, geldanamycin, methotrexate, camptothecin, paclitaxel, gemcitabine, temozolomide, cyclophosphamide, cyclosporine, a non-steroidal anti-inflammatory drug, a cytokine suppressive anti-inflammatory drug, a corticosteroid, methotrexate, prednisone, cyclosporine, morroniside cinnamic acid, leflunomide, and a combination thereof.

Exemplary anti-diabetic agents include insulin, exenatide, liraglutide, pramlintide, biguanides such as metformin; sulfonylureas such as glyburide or glimepiride; meglitinides such as nateglinide or repalinide; DPP-4 inhibitors such as saxagliptin or sitagliptin; GLP-1 agonists such as the incretin mimetic drugs: exenatide, liraglutide, albiglutide; SGLT-2 inhibitors such as canagliflozin, dapaglifozin, and empagliflozin; alpha-glucosidase inhibits such as acarbose; thiazolidinediones such as pioglitazone; and amylin analogs such as pramlintide.

Exemplary anti-cancer agents include a cytotoxic agent, an alkylating agent, an antineoplastic agent, an antiproliferative agent, an antitubulin agent, a chemotherapeutic agent, a toxin, auristatin, a DNA minor groove binding agent, a DNA minor groove alkylating agent, a 5-ipoxygenase inhibitor, or a leukotriene receptor antagonist, an enediyne, a lexitropsin, a duocarmycin, a taxane, a puromycin, a dolastatin, a maytansinoid, and a vinca alkaloid.

Exemplary anti-cancer agents include aldosterone, amrubicin, an auristatin, azathioprine, biricodar, bleomycin, busulfan, camptothecin, carboplatin, carmustine, chlorambucil, cisplatin, cyclophosphamide, cyclosporine, cytarabine, cytochalasin B, cytosine arabinoside, dactinomycin, daunorubicin, dexamethasone, docetaxel, doxorubicin, emetine, epirubicin, etanercept, etoposide, 5-fluorouracil, floxuridine, gancyclovir, gemcitabine, gramicidin D, idarubicin, irinotecan, lomustine, mechlorethamine, melphalan, 6-mercaptopurine, methotrexate, mycophenolate mofetil, mithramycin, a mitomycin, mitoxantrone, oxaliplatin, paclitaxel, pirarubicin, plicamycin, probenecid, puromycin, raloxifene, rapamycin, ricin, tacrolimus, tamoxifen, taxol, teniposide, thalidomide, 6-thioguanine, thiotepa, topotecan, verapamil, vinblastine, vincristine, vindesine, vinorelbine, analogs thereof and combinations thereof.

Exemplary hormones and analogs thereof include estrogens, antiestrogens, progestins, androgens, antiandrogens, such as corticosterone, cortisol, dihydroxytestosterone, estradiol, estrone, progesterone, testosterone and the like.

Examples of small molecule active agents include doxorubicin, paclitaxel, gemcitabine, camptothecin, temozolomide, and the like. Examples of suitable peptidic drugs include insulin, GLP-1, Exendin-4, octreotide, bombesin, RGD peptide (arginylglycylaspartic acid), and the like, or a therapeutic fragment thereof. Examples of suitable therapeutic proteins include vascular endothelial growth factor (VEGF), interferon (IFN), tumor necrosis factor (TNF), asparaginase, adenosine deaminase, and the like, or a therapeutic fragment thereof. Another example of a useful therapeutic peptide that may be included in the compounds and methods described herein is Exendin (9-39), a 31 amino acid fragment of Exenatide which is useful, for example, in the treatment of post-bariatric hypoglycemia. Preferably, the target molecule included as a conjugate in Formula I can treat or diagnose diseases or conditions in mammals, preferably humans. For example, R₁₃ can be selected for its ability to treat or diagnose cancer or diabetes. Marker compounds, often referred to as marker molecules, include fluorescent tags, often referred to as fluorescent agents, and comprise a fluorophore; and a bioluminescent molecules (e.g., luciferase). Fluorescent agents include fluorescein isothiocyanate (FITC), allophycocyanin (APC), phycoerythrin (PE), rhodamine, tetramethyl rhodamine isothiocyanate (TRITC), fluorescent protein (GFP), enhanced GFP (eGFP), yellow fluorescent protein (YFP), cyan fluorescent protein (CFP), red fluorescent protein (RFP), or dsRed.

Radioactive agents include agents labeled with ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ⁶¹Cu, ⁵³Gd, ¹²⁵I, ¹³¹I and ³²P, suitable for use with positron emission tomography (PET) and single photon emission computed tomography (SPECT) imaging procedures.

The contrast agent can be for medical imaging. In an embodiment, the contrast agent is for magnetic resonance imaging (MRI) and is a gadolinium-containing compound such as gadodiamide (OMNISCAN), gadobenic acid (MULTIHANCE), gadopentetic acid (MAGNEVIST), or gadoteridol (PROHANCE).

In one embodiment, the R₁₃ is a quantum dot. Quantum dots, nanocrystalline semiconductor materials, are composed of periodic groups II-VI, III-V, or IV-VI materials. The diameter of the quantum dots can range from about 1 to about 20 nanometers. Exemplary quantum dots include cadmium selenide/zinc sulfide core-shell nanocrystals.

In another embodiment the R₁₃ group is a liposome or a liposome precursor. The liposomes can be surface-modified by covalently bonding the cyclic peptide to the liposome or a liposome precursor. Exemplary liposomes include nanoscale unilamellar liposomes and polymerized liposomal nanoparticles (PLNs). The liposome covalently bonded to the cyclic peptide can be used to encapsulate a pharmaceutically active agent or diagnostic agent such as those previously described herein.

Proteins or peptides include hormones, cytokines, growth factors, clotting factors, anticoagulants, bacterial or plant toxins, drug-activating enzymes, antibodies, peptides, and peptidomimetics. The cytokine can be, for example, tumor necrosis factor α (TNF), interferon gamma, interferon α, endostatin, or tumstatin.

Exemplary nucleic acids are an anti-sense nucleic acid, a small interfering RNA (siRNA,) a microRNA (miRNA), a peptide nucleic acid (PNA), and a locked nucleic acid (LNA). The nucleic acid complex can be a viral particle, or a recombinant viral vector such as an adenoviral or adeno-associated viral vector.

In certain embodiments, R₁₃ is insulin, an insulin analog, IL-2, IL-5, GLP-1, BNP, IL 1-RA, KGF, ancestim, GH, G-CSF, CTLA-4, myostatin, Factor VII, Factor VIII, Factor IX, Exendin-4, exendin (9-39), octreotide, bombesin, RGD peptide (arginylglycylaspartic acid), vascular endothelial growth factor (VEGF), interferon (IFN), tumor necrosis factor (TNF), asparaginase, adenosine deaminase, a therapeutic fragment of any of the foregoing, a derivative of any of the foregoing, calicheamycin, auristatin, doxorubicin, maytansinoid, taxane, ecteinascidin, geldanamycin, methotrexate, camptothecin, paclitaxel, gemcitabine, temozolomide, cyclophosphamide, cyclosporine, a non-steroidal anti-inflammatory drug, a cytokine suppressive anti-inflammatory drug, a corticosteroid, methotrexate, prednisone, cyclosporine, morroniside cinnamic acid, leflunomide, or a combination thereof.

R₁₃ can be a native therapeutic polypeptide, or a therapeutically active fragment thereof. Preferably, R₁₃ contains a sulfhydryl moiety that facilitates conjugation or cross-linking between it and the crosslinking moiety of Formula I, such as a maleiminde or thiol, to form the conjugate. The active sulfhydryl moiety on the therapeutic compound may be naturally occurring (for example, Exendin-4 includes a cysteine (Cys) residue at position 40, herein exendin-4 may also be denoted as Cys40-exendin), or may be artificially introduced into the therapeutic compound or fragment by methods well known in the art such as amino acid substitution or chemical modification.

The compounds comprising a triazole ring as one of the linking groups can be prepared using azide-alkyne Huisgen 1,3-dipolar cycloaddition reaction (“Click” chemistry). The use of Click chemistry allows for the convenient synthesis of a wide array of conjugates. The resulting triazole unit formed from the cycloaddition is less likely to be attacked by hydrolytic enzymes and esterases compared to typical amide or ester bonds.

One precursor of the conjugate comprises an azide group and the second precursor comprises an alkyne group, e.g., a terminal alkyne. The conjugation of the two precursor molecules can be effected using copper(I)-catalyzed azide-alkyne cycloaddition which results in 1,4-regioisomers of 1,2,3-triazoles as sole products. Exemplary copper(I) catalysts for use in the reaction include cuprous bromide, cuprous iodide, or a mixture of a copper(II) compound (e.g. copper(II) sulfate) and a reducing agent (e.g. sodium ascorbate) to produce a copper(I) catalyst in situ. A reducing agent can be employed in the reactions using copper(I) catalyst. Alternatively, the conjugation can be effected using a cyclopentadienyl(Cp)*Ru(II) catalyst, such as pentamethyl cyclopentadienyl bis(triphenylphosphine)ruthenium(II) ((Cp)*Ru(PPh₃)₂), to yield 1,5-substituted 1,2,3-triazoles as sole products.

Exemplary compounds comprising an azide group suitable for preparing the azide-containing precursor include azide containing carboxylic acids such as 2-azido acetic acid, 3-azidopropanoic acid, 4-azidobutanoic acid, and the like.

Suitable solvents for conducting the cycloaddition reaction are those that do not adversely affect the reaction, and specifically are inert. Suitable solvents can further be selected on the basis of economics, environmental factors, and the like, and may be organic, aqueous, or a mixture thereof. Suitable organic solvents may be aliphatic alcohols such as methanol, ethanol, n-propanol, isopropanol, tert-butanol, n-butanol, and the like; aliphatic ketones such as acetone and methyl ethyl ketone; aliphatic amide such as dimethylformamide or dimethylacetamide; aliphatic carboxylic esters such as ethyl acetate; aromatic hydrocarbons such as toluene and xylene; aliphatic hydrocarbons such as hexane; aliphatic nitriles such as acetonitrile; chlorinated hydrocarbons such as dichloromethane; aliphatic sulfoxides such as dimethyl sulfoxide; aliphatic and cyclic ethers such as tetrahydrofuran; aqueous mixtures of water and a miscible or partially miscible organic solvent, specifically in combination with a stabilizing ligand (e.g., tris-(benzyltriazolylmethyl)amine (TBTA)); and the like, as well as combinations thereof.

In Formula I, B is v,

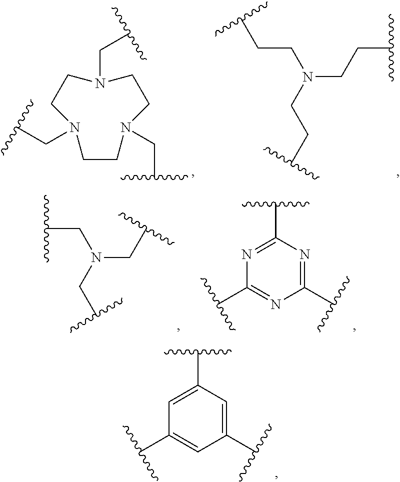

—(CH₂)n- wherein n is an integer from 0 to 12, wherein each CH₂ can be individually replaced with —O—, —NH(CO)—, or —(CO)NH— providing no two adjacent CH₂ groups are replaced, and wherein —(CH₂)n- is substituted with one substituent A₃.

In certain embodiments of the compound of Formula I, A₁ is —NH(CS)NH—, A₂ is —(CO)NH—, and B is —(CH₂)₄(CHA₃)— wherein A₃ is (-L₂R₁₂) and L₂ is —NH(CO)CH₂—. Preferably R₁₂ is

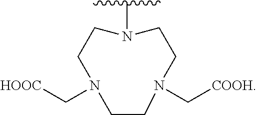 [SMILES: *N1CCN(CC(=O)O)CCN(CC(=O)O)CC1]

In certain embodiments of the compound of Formula I, A₁ is —NH(CH₂)ₘ(CO)— and A₂ is —(CO)(CH₂)ₖNH— wherein independently each of m and k is an integer from 0 to 4, and B is

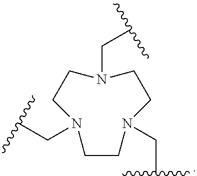 [SMILES: *CN1CCN(C*)CCN(C*)CC1]

Preferably each of m and k is independently an integer from 0 to 2, more preferably m=0, k=0. A₃ can be —COOH or -L₂-R₁₂. When A₃ is -L₂-R₁₂, the linking group L₂ is preferably —[(CO)NH(CH₂)r]-, r is an integer from 1 to 3, and R₁₂ is a crosslinker or a conjugate, preferably R₁₂ is

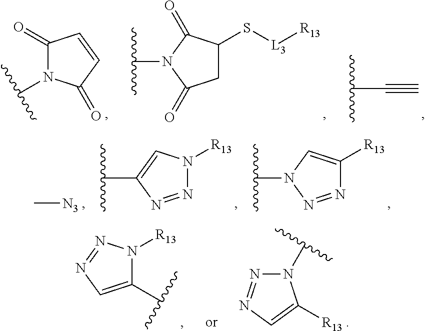

R₁₃ is a target molecule, as described elsewhere herein. The linking group L₃ is —(CH₂)_{q}— wherein q is an integer from 0 to 12, and each CH₂ can be individually replaced with —O—, —NH(CO)—, or —(CO)—NH—, providing no two adjacent CH₂ groups are replaced.

In embodiments of the compound of Formula I, B is

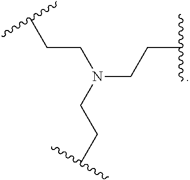 [SMILES: *CCN(CC*)CC*]

In certain of these embodiments A₁ is —NH(CS)NH—, A₂ is —NH(CS)NH—, and A₃ is —NH₂ or -L₂-R₁₂. In other of these embodiments, A₁ is —NH(CO)—, A₂ is —(CO)NH—, and A₃ is —COOH or -L₂-R₁₂. When A₃ is -L₂-R₁₂, R₁₂ is a crosslinker or a conjugate, preferably R₁₂ is

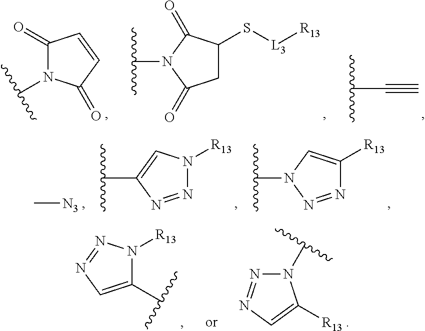

R₁₃ is a target molecule as described herein. The linking group L₃ is —(CH₂)_{q}— wherein q is an integer from 0 to 12, and each CH₂ can be individually replaced with —O—, —NH(CO)—, or —(CO)—NH—, providing no two adjacent CH₂ groups are replaced.

In embodiments of the compound of Formula I, B is

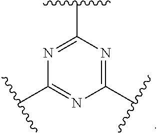 [SMILES: *c1nc(*)nc(*)n1]

In preferred embodiments A₁ is

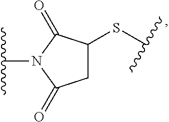 [SMILES: *SC1CC(=O)N(*)C1=O]

### A₂ is

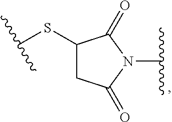 [SMILES: *SC1CC(=O)N(*)C1=O]

and A₃ is —SH or -L₂-R₁₂. In preferred embodiments A₁ is —NH—, A₂ is —NH—, and A₃ is —Cl or -L₂-R₁₂. In preferred embodiments A₁ is —NH(CO)—, A₂ is —(CO)NH—, and A₃ is —COOH or -L₂-R₁₂. When A₃ is -L₂-R₁₂, R₁₂ is a crosslinker or a conjugate, preferably R₁₂ is

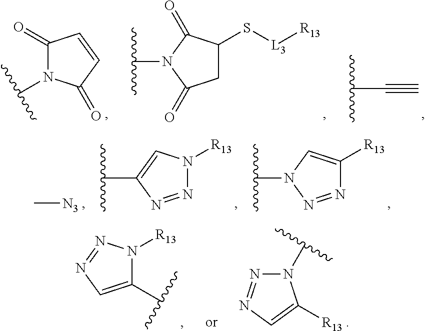

R₃ is a target molecule as described herein. The linking group L₃ is —(CH₂)_{q}— wherein q is an integer from 0 to 12, and each CH₂ can be individually replaced with —O—, —NH(CO)—, or —(CO)—NH—, providing no two adjacent CH₂ groups are replaced.

Preferred compounds of Formula I include

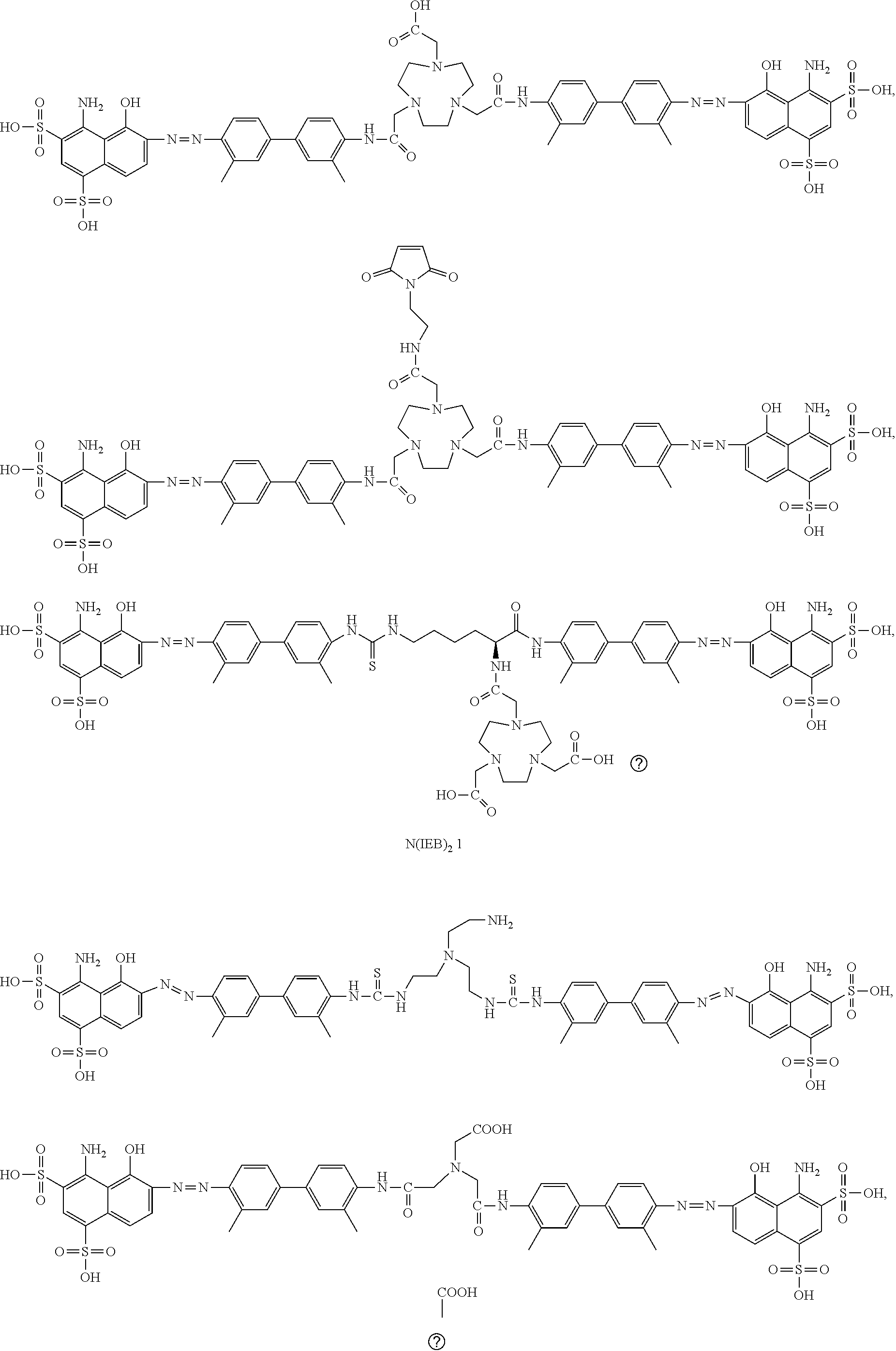

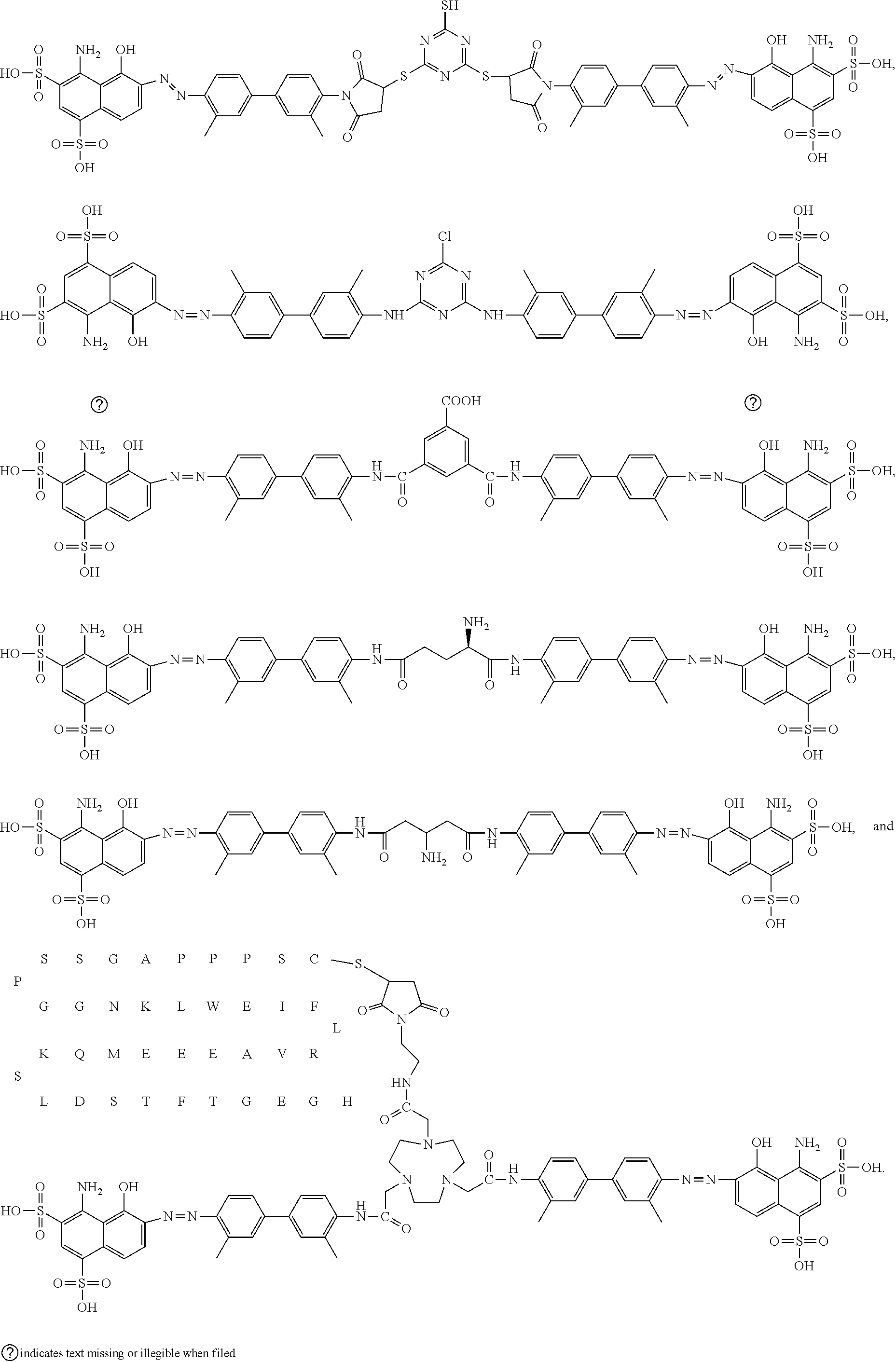

In the compound of Formula I, R₁₂ can further comprise a radionuclide. The radionuclide can be ¹⁸F, ⁷⁶Br, ¹²⁴I, ¹²⁵I, ¹³¹I, ⁶⁴Cu, ⁶⁷Cu, ⁹⁰Y, ⁸⁶Y, ¹¹¹In, ¹⁸⁶Re, ¹⁸⁸Re, ⁸⁹Zr, ⁹⁹Tc, ¹⁵³Sm, ²¹³Bi, ²²⁵Ac, ¹⁷⁷Lu, ²²³Ra, or a combination thereof. These compounds are useful as imaging agents or as reagents in diagnostic assays. The radionuclide may be bound to R₁₂ by chelation, or by other means such as conventional covalent or ionic bonds known in the chemical arts. The radionuclide may be suitable for purposes such as imaging or scanning, for example PET imaging, and the compound of Formula I may be a PET imaging agent. The radionuclide may be suitable for purposes of patient treatment, for example radiation treatment.

Compositions and Pharmaceutical Preparations

Reference to a formula includes references to all subformulae. Compounds disclosed herein can be administered as the neat chemical, but are preferably administered as a composition, more preferably a pharmaceutical compositions.

Accordingly, also disclosed are compositions comprising a compound or pharmaceutically acceptable salt of a compound disclosed herein, such as a compound of Formula I, together with at least one carrier, preferably a pharmaceutically acceptable carrier. The composition may contain a compound disclosed herein as the only active agent, but is preferably contains at least one additional active agent. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition may also include a molar ratio of a compound, such as a compound of Formula I, and an additional active agent. For example the pharmaceutical composition may contain a molar ratio of about 0.5:1, about 1:1, about 2:1, about 3:1 or from about 1.5:1 to about 4:1 of an additional active agent to a compound of Formula I.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the compound of the present invention.

The pharmaceutical compositions/combinations can be formulated for oral administration. These compositions contain between 0.1 and 99 weight % (wt. %) of a compound of Formula I and usually at least about 5 wt. % of a compound of Formula I. Some embodiments contain from about 25 wt % to about 50 wt % or from about 5 wt % to about 75 wt % of the compound of Formula I.

Methods of Use

The compounds of Formula I, as well as pharmaceutical compositions comprising the compounds, are useful for diagnosis or treatment of diseases such as diabetes or cancer. In an embodiment, a method of treating diabetes comprises providing to a patient in need of such treatment a therapeutically effective amount of a compound of Formula I. Preferably, in the compound of Formula I, R₁₂ is a chelating group or a conjugate. The compounds of Formula I provided herein may be administered alone, or in combination with one or more other active agents. In an embodiment, the patient is a mammal. The mammal can be a human, a companion animal, for example a cat or dog, a horse, or livestock, e.g. cattle, sheep, cows, goats, swine, and the like. Preferably the mammal is a human.

A therapeutically effective amount of a compound or a composition disclosed herein is an amount sufficient to reduce or ameliorate the symptoms of a disease or condition. In the case of diabetes for example, a therapeutically effective amount may be an amount sufficient to reduce or ameliorate high blood sugar. A therapeutically effective amount of a compound or pharmaceutical composition described herein will also provide a sufficient concentration of a compound of Formula I when administered to a patient. A sufficient concentration is preferably a concentration of the compound in the patient's body necessary to prevent or combat the disorder. Such an amount may be ascertained experimentally, for example by assaying blood concentration of the compound, or theoretically, by calculating bioavailability.

The methods of treatment disclosed herein include providing certain dosage amounts of a compound of Formula I to a patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I are provided daily to a patient. Frequency of dosage may also vary depending on the compound used and the particular disease treated. However, for treatment of most diseases and disorders, a dosage regimen of 4 times daily or less can be used and in certain embodiments a dosage regimen of 1 or 2 times daily is used. It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

A compound of Formula I may be administered alone (i.e., as sole therapeutic agent of a regime) to treat or prevent diseases and conditions such as diabetes, or may be administered in combination with another active agent. One or more compounds of Formula I may be administered in coordination with a regime of one or more other active agents such as insulin secretagogs.

For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g. blood, plasma, serum, cellular interstitial fluid, saliva, feces, and urine) and cell and tissue samples of the above subjects will be suitable for use.

In an embodiment, the method of treating diabetes may additionally comprise administering the compound of Formula I in combination with one or more additional compounds, wherein at least one of the additional compounds is an active agent, to a patient in need of such treatment. The one or more additional compounds may include insulin, exenatide, DPP-4 (dipeptidyl peptidase-4) inhibitors, neuropilin, EGF (epidermal growth factor), INGAP (islet neogenesis associated protein), alpha-1 antitrypsin, anti-inflammatory agents, glulisine, glucagons, local cytokines, modulators of cytokines, anti-apoptotic molecules, aptamers, asparaginase, adenosine deaminase, interferon α2a, interferon α2b, G-CSF (granulocyte colony stimulating factor), growth hormone receptor antagonists, and combinations thereof.

In another aspect, a method of increasing the in vivo half-life of a target molecule is disclosed. The method comprises covalently coupling a compound of Formula I disclosed herein to the target molecule. Preferably in the compound of Formula I, R₁₂ is a crosslinker. Examples of the target molecule have been disclosed elsewhere herein.

In another aspect, a method of in vivo imaging is disclosed. The method comprises administering to a subject a compound of Formula I. Preferably in the compound of Formula I, R₁₂ is a chelating group or a conjugate. The method further comprises imaging the subject. Examples of imaging methods include PET and fluorescence imaging.

The compositions of the present invention offer the advantage that many small molecules and biologics can be easily modified in one step with high yield and high purity. Due to the relatively strong binding of EB moiety with albumin, the in vivo biodistribution can be easily controlled to adjust the number of EB moieties and linkers. In addition, the relative small size of the EB moiety reduces the likelihood of any interference with the biological function of the small molecule or biologic. The addition of a chelator, such as NOTA or DOTA linked to the EB moiety allows for facile addition of further groups such as radionuclides, which can allow the present molecules to act as imaging agents and/or radiotherapeutic agents. The present invention therefore provides an efficient system for developing long lasting and long acting therapeutic and imaging agents with high efficacy.

This disclosure is further illustrated by the following examples, which are non-limiting.

### EXAMPLES

All parts and percentages are by weight and all temperatures are degrees Celsius unless explicitly stated otherwise.

Qualitative Liquid Chromatography/Mass Spectrometry (LC/MS)

Waters Liquid Chromatography/Mass Spectrometry (LC/MS) system (Waters, Milford, Mass.) was employed with an Acquity UPLC system coupled to the Waters Q-Tof Premier high-resolution mass spectrometer. An Acquity BEH Shield RP18 column (150 mm×2.1 mm) was eluted with a two-solution gradient of solution A (2 mM ammonium formate, 0.1% formic acid, and 5% CH₃CN) and solution B (2 mM ammonium formate and 0.1% formic acid in CH₃CN). The elution profile at 0.2 mL/min was as follows: 100% (v/v) A and 0% B initially, gradient from 0 to 40% B over 5 min, isocratic elution at 40% B for an additional 5 min, washing with 100% B over 2 min, and re-equilibrium with A for an additional 4 min. The injection volume was 10 μL. The entire column elute was introduced into the Q-Tof mass spectrometer. Ion detection was achieved in electrospray ionization (ESI) mode using a source capillary voltage of 3.5 kV, source temperature of 100° C., desolvation temperature of 200° C., cone gas flow of 50 L/h (N₂), and desolvation gas flow of 700 L/h (N₂).

Cell Culture

U-87MG (human glioblastoma) and INS-1 (rat insulinoma) were purchased from American Type Culture Collection (ATCC, Rockville, Md.), and UM-22B (human head and neck squamous carcinoma) cells were purchased from EMD Millipore (Billerica, Mass.). The cells were cultured in Minimum Essential Medium (MEM), RPMI-1640 medium, and Dulbecco's modified Eagle medium (DMEM) respectively, containing 10% fetal bovine serum (Gibco) in Acell incubator (a humidified atmosphere containing 5% CO₂ at 37° C.). The cells were passaged 2-3 times per week.

Animal Models

All animal protocols were approved by the NIH Clinical Center Animal Care and Use Committee (ACUC). The studies for in vivo pharmacokinetics and lymph node mapping were performed in normal BALB/c mice (female; age, 6-8 weeks; weight, 18-20 g) (Harlan).

For mouse xenografts model, female nude mice (6-8 weeks, 20-23 g) (Harlan) were inoculated on their right shoulder with 5×10⁶ cells of U-87MG, INS-1 or UM-22B cells in Matrigel (Sigma) and PBS with volume ratio of 1:1, respectively. The mice underwent small-animal PET studies when the tumor volume reached 100-300 mm³ (2-3 wk after inoculation).

### Example 1: Computational Modeling of Evans Blue Derivatives and Albumin Binding Site

Evans Blue and EB Derivatives Bind to Cleft and Site II on Albumin

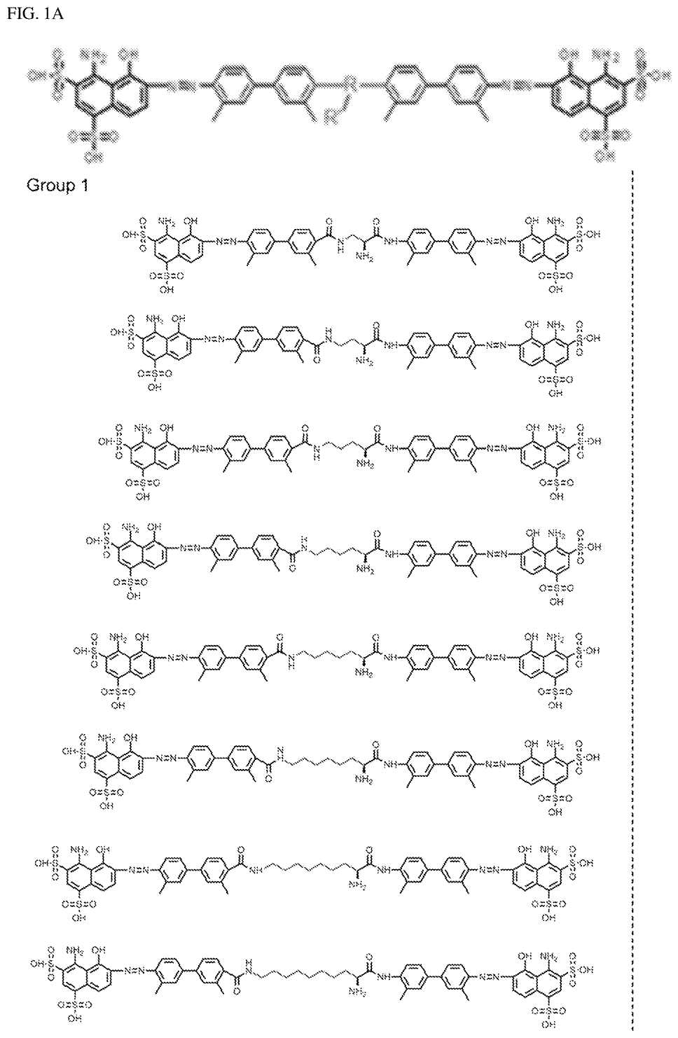

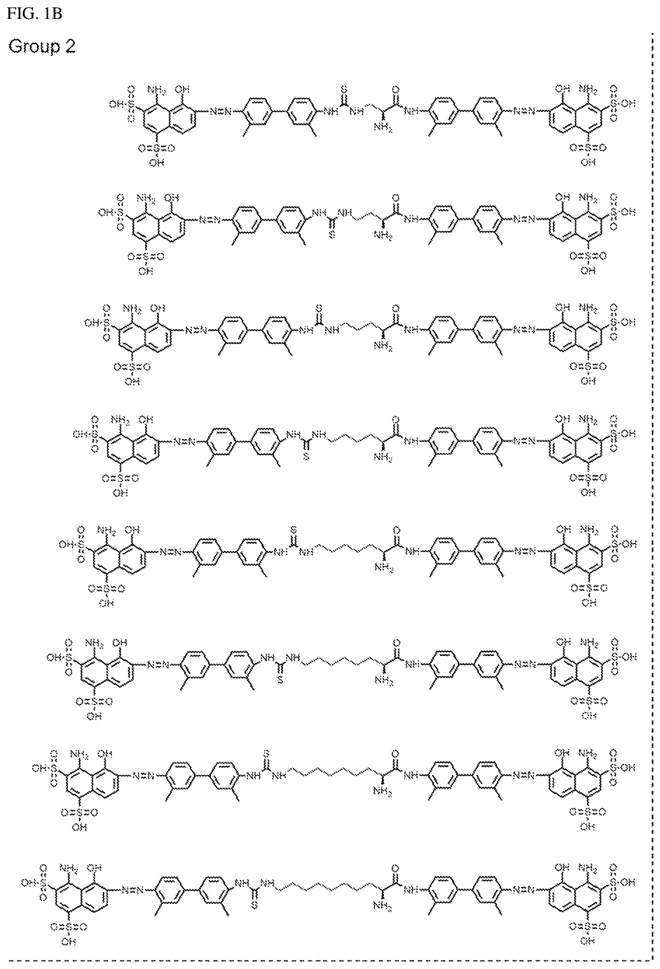

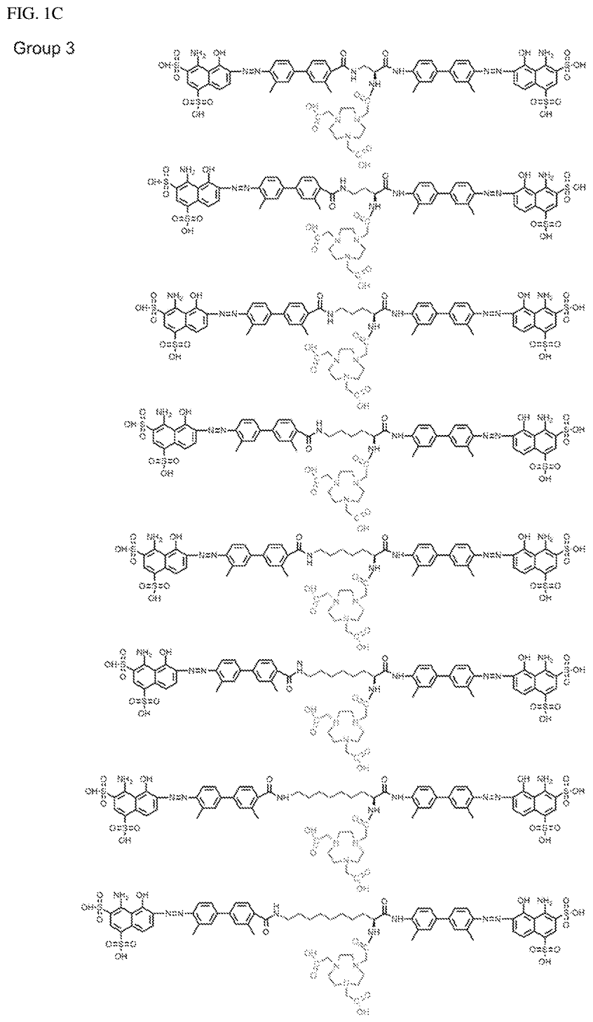

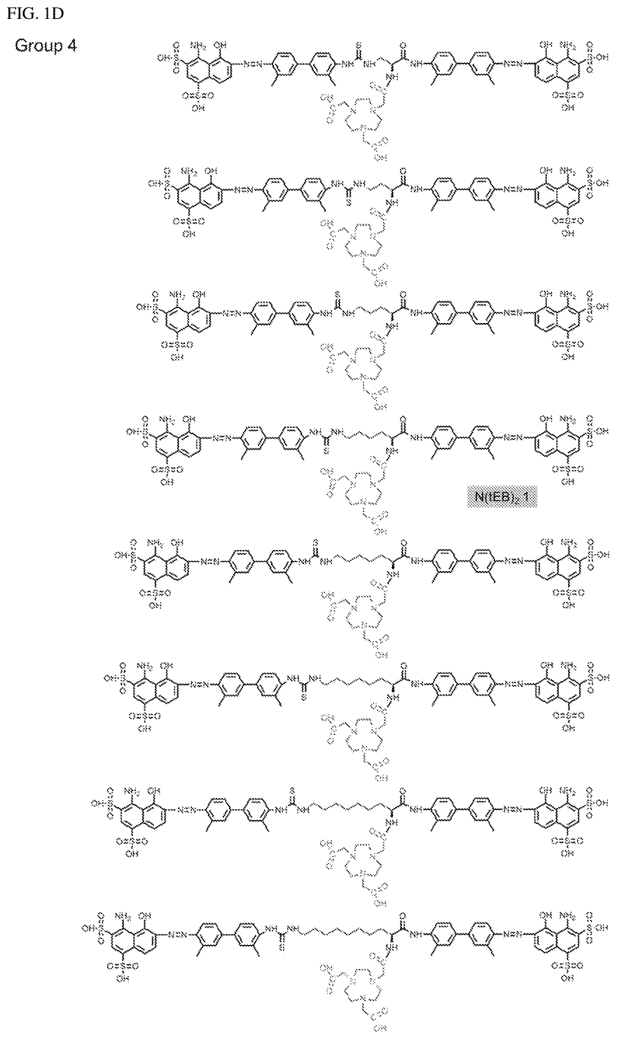

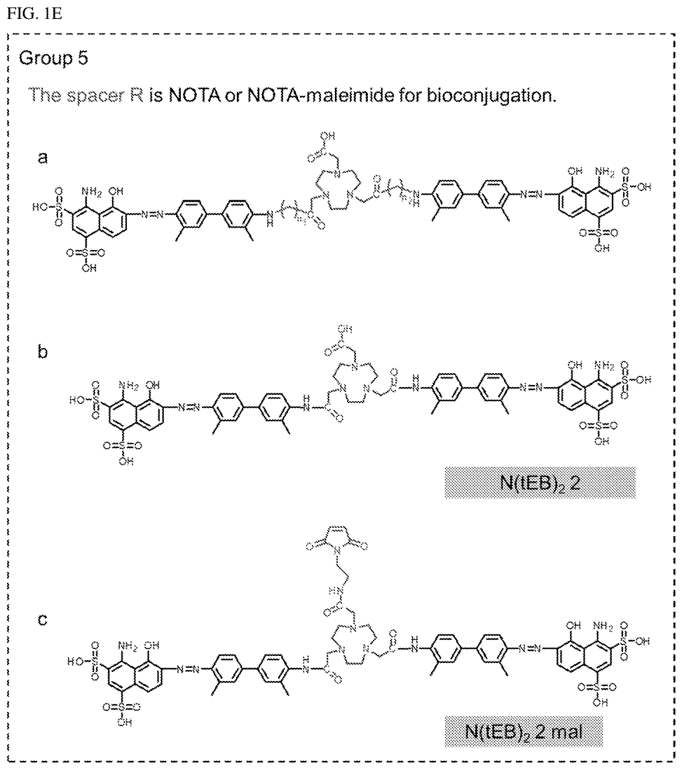

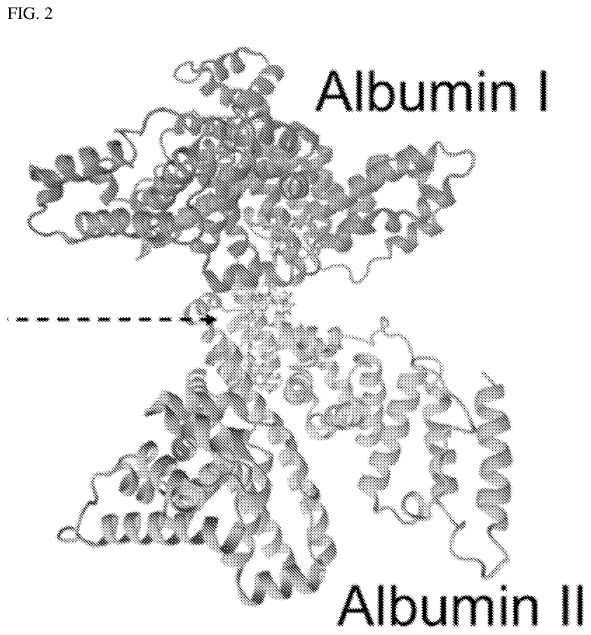

Multivalency is an effective strategy to increase the interaction of individual ligands with their respective receptors. We thus constructed a virtual library of tEB dimers ((tEB)₂) with different linkers (FIGS. 1A-E) and screened the library based on computational modeling. With two albumin binding motifs, (tEB)₂ was expected to bind two albumin molecules and form a reversible albumin-(tEB)₂-albumin sandwich structure (FIG. 2). We hypothesized, this in vivo dimerization would result in enhanced tumor retention after intravenous injection and delayed lymphatic migration after subcutaneous injection of (tEB)₂ compared with previous EB constructs with only one binding moiety. Furthermore, this albumin-dimer will create a cavity (see FIG. 2) whereby a conjugated therapeutic small molecule or peptide can be protected from enzymatic degradation.

As shown by the generic structure at the top of FIG. 1A, the various N(tEB)₂ in the library have two albumin binding moieties, the 4-amino-5-hydroxynaphthalene-1,3-disulfonic acid group, a spacer (R) joining the two tEB monomers via the terminal phenyl rings, and/or a side-chain chelator (R′). The spacer (R) is tunable in length, such as different lengths of aliphatic chains between the two reactive linker ends, which can be the same or different. R′ is a hydrogen or a moiety for conjugating to an active compound, such as a drug, or chelating an isotope to enable radiolabeling and imaging. The moiety for chelating an isotope can be a 1,4,7-triazacyclononane-1,4,7-trisacetic acid (NOTA) group, while the moiety for conjugating drugs can be, for example, a thiol or a NOTA derivatized with a reactive moiety such as a maleimide or a thiol. In FIGS. 1A-1D, group 1 shows the structures of the (tEB)₂ with an aliphatic chain as a spacer (1≤n≤8); group 2 shows the structures of (tEB)₂ molecules with an additional thiourea group on the aliphatic chain (1≤n≤8); group 3 shows the structures of (tEB)₂ molecules having a NOTA group on the aliphatic chain (1≤n≤8); and group 4 shows the structures of (tEB)₂ molecules in which both a thiourea group and a NOTA group were introduced into the spacer with different lengths of the aliphatic chains (1≤n≤8). In FIG. 1E, the group 5 structures of N(tEB)₂ were designed to include a NOTA group as a center linker in the backbone of the spacer rather than as a side-chain chelator on the aliphatic chain. In the Group 5 generic structure (a), the spacer (R) is tunable with length in the center with different lengths of aliphatic chains (n₁≤2, n₂≤2). The optimal length of the structures represented by generic structure (a), determined by the computational modeling, is shown in FIG. 1E panel b and has n₁=0, n₂=0. This molecule was designated “N(tNEB)₂ 2”. A final molecule modeled included a maleimide group conjugated with the NOTA group of N(tNEB)₂ 2, denoted “N(tNEB)₂ 2-mal” as shown in FIG. 1E, panel c, for conjugation with thiol-containing small molecules.

The derivatized N(tEB)₂ 2 was designed for conjugation with functional molecules for two reasons. First, N(tEB)₂ 2 showed lowest free energy in the computations and second, compared with the NOTA group as a side chain on the aliphatic chain, the rigid conformation and relatively short branch of the maleimide-derivatized N(tEB)₂ 2 kept the functional cargo within the cleft so that the attached small molecules can deeply insert into the cleft and be protected from degradation.

We computationally modeled the binding sites for the albumin binding motifs, using the open source software package AutoDock Vina (O. Trott, A. J. Olson, AutoDock Vina: improving the speed and accuracy of docking with a new scoring function, efficient optimization and multithreading, Journal of Computational Chemistry 31 (2010) 455-461) to generate the docking poses. The center cleft on albumin was found to be the most preferable binding site, followed by site II. Our results ran contrary to previously published literature that EB binds preferentially to site I. To substantiate this finding, we incubated the recombinant HSA subunits (three separate domains of albumin) with EB and performed high-resolution liquid chromatography-mass spectrometry (LC-MS) to detect complex formation between the individual recombinant HSA subunits with EB. Only domain III subunit, which contains site II, was observed to complex with EB by LC-MS. Collectively, these data indicated the unexplored cleft and/or site II on domain III rather than site I on domain II A was the binding site(s) for EB and its derivatives.

Design and Optimization of N(tEB)₂ with Modeling Simulation

After confirming the binding sites of EB in albumin, we designed a library of derivatives with two albumin binding motifs and linkers with variable lengths, ranging from 5.2-10.8 Å. Docking simulation and protein-protein interaction analysis were conducted to screen for optimal binders. A prospective structure (tEB)₂ was first constructed, with either an aliphatic chain or a NOTA group in the center (FIGS. 1A-C). The (tEB)₂ with only an aliphatic chain as the linker was extremely flexible and tended to fold, so it was unable to bind two albumin molecules. A thiourea group was introduced to promote intramolecular hydrogen bond formation with the NOTA group, and to stabilize the rigid conformation of (tEB)₂ molecule. Next, we screened the rigid-confirmation scaffolds listed in FIGS. 1A-C, with different aliphatic chain lengths (1≤n≤8) to determine the optimal distance for albumin binding. We evaluated the albumin interaction through the molecular mechanics/Poisson-Boltzmann surface area (MM/PBSA) analysis on five 1-ns conformation sampling trajectories of the proteins with distances of 60/70/80/90/100 between their centers of mass. The distance range of 64-80 Å (minimum distance of 4.7-16.4 Å from edge to edge) was found to be the best for increasing albumin-albumin intermolecular attraction and avoiding steric hindrance between the two adjacent albumin molecules, indicating that the scaffolds with an aliphatic chain consisting of 3 to 5 methylene groups were optimal for dual albumin binding. Alternatively, when the NOTA group was engineered as part of the backbone (FIG. 1E) rather than as a side-chain moiety on the aliphatic chain, we also observed restricted self-folding of (tEB)₂.

When N(tEB)₂ and EB were placed in a cubic TIP3P water model with a buffer space 12 Å on each side using both the parallel and angular starting poses, the free EB dye showed strong tendency to form π-π stacking. N(tEB)₂ avoided intermolecular stacking and self-assembly. The results from absolute binding free energy calculations between the N(tEB)₂ and albumin(s) via a double-decoupling scheme confirmed that the most stable complex for the mixture of N(tEB)₂ and albumin molecules was the “sandwich” HSA-N(tEB)₂—HSA dimer with ΔG_{bind} of −22.3 kcal/mol), which was far less than that of N(tEB)₂—HSA monomer (ΔG_{bind}=−7.1 kcal/mol). The data suggests that this optimized configuration allows for binding cooperativity and overcomes steric hindrance for binding two albumins.

### Example 2: Synthesis of N(tEB)₂ 1

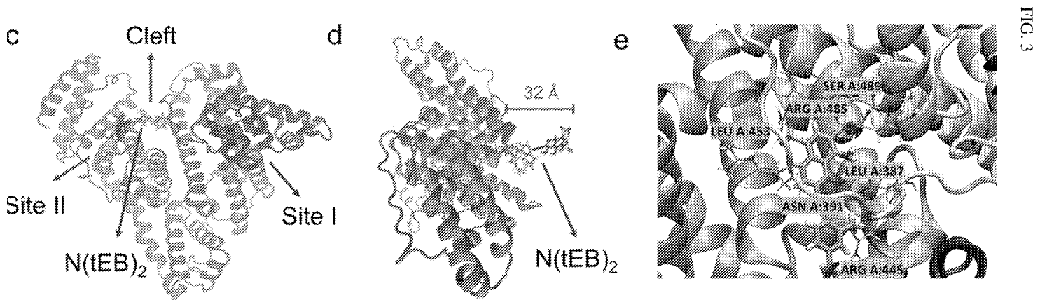

The chemical synthesis of N(tEB)₂1 followed the synthetic scheme in FIGS. 4A-4C.

In detail, 4.25 g o-tolidine (compound 1) (4.25 g, 20.0 mmol) and 50 mL dichloromethane were added to a 250 mL glass vial, and then 20 mL di-tert-butyl dicarbonate (4.36 g, 20.0 mmol) in dichloromethane was added dropwise to the vial. The mixture was stirred in room temperature (RT) for 24 h, then the solvents were removed under reduced pressure, and the residuum was purified by silicon column to obtain the compound 2. To 3.12 g compound 2 in 40 mL water was added 15 mL 2 M HCl, after cooled in ice bath, 20 mL NaNO₂ (2.07 g, 30.0 mmol) was added, the mixture was stirred in ice bath for 20 mins and yellow diazonium salt solution was formed (compound 3). NaHCO₃ (3.36 g) was added to 3.19 g 1-amino-8-naphthol-2,4-disulfonic acid in 20 mL water, then compound 3 solutions was added dropwise and the mixture was stirred in ice bath for 2 h. The solvents were removed under reduced pressure, and the residuum was purified by C18 column to obtain the compound 4. Compound 4 (3.22 g) was added to 20 mL Trifluoroacetic acid (TFA) in batches, stirred in RT for 60 min, and then the solvents were removed under reduced pressure, and the residuum was purified by C18 column to obtain the compound 5. Ammonium hydroxide (1 mL) was added dropwise to 1.08 g compound 5 in dimethyl formamide (DMF), followed by stirred at RT overnight. Then, 0.74 g CS₂ was added to the mixture and stirred at 40° C. for 8 h. the residuum was purified by C18 column to obtain the compound 6. The purified compound 6 (0.58 g) and Pb(NO₃)₂ (0.66 g) was mixed in 50 mL acetonitrile overnight at RT. The mixture was purified by High Performance Liquid Chromatography (HPLC) to get the compound 7.

Compound 5 (0.54 g), compound 8 (0.43 g), 1-[Bis(dimethylamino)methylene]-1H-1,2,3-triazolo[4,5-b]pyridinium 3-oxid hexafluorophosphate (HATU) (0.38 g) and diisopropylethylamine (DIPEA) (0.26 g) were added to 20 mL DMF, and the mixture was stirred at RT for 24 h. The solvents were removed under reduced pressure, and the residuum was purified by C18 column to obtain the compound 9. To a solution of compound 9 (0.48 g) in 10 mL DMF, 2 mL piperidine was added dropwise in ice bath, and the mixture was stirred for 2 h at RT. The solvents were removed under reduced pressure, and the residuum was purified by C18 column to obtain the compound 10. Then, 0.37 g of the compound 10, 0.25 g NOTA-(OtBu)₂ (“OtBu” is tertbutyl ester), 0.19 gHATU and 0.13 g DIPEA was in turn added to 5 mL DMF. The mixture was stirred at room temperature for 24 h, and then the solvents were removed under reduced pressure. The residuum was purified by C18 column to obtain the compound 11. A solution of compound 11 (0.24 g) in 20 mL TFA was stirred for 2 h at RT, the solvent was removed under reduced pressure and compound 12 was acquired by C18 column.

Finally, compound 7 (0.06 g), compound 12 (0.1 g), and DIPEA (0.04 g) was added to 2 mL DMF, the mixture was reacted at room temperature for 24 h, and the (N(EB)₂) 1 was purified and freeze dried. The exact molecular weight of N(tEB)₂ 1 was 1539.4 g/mol.

### Example 3: Synthesis of N(tEB)₂ 2 and N(tEB)₂ 2-Mal

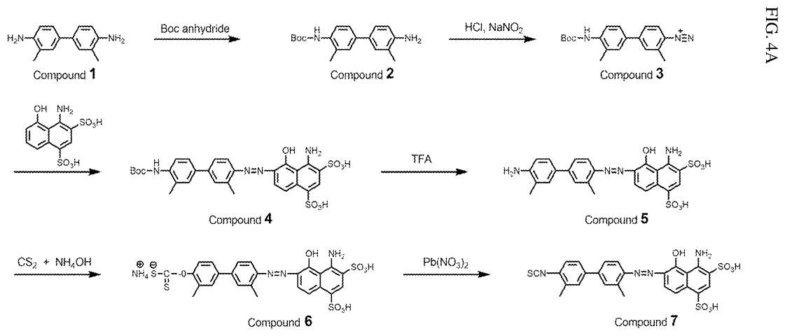

The chemical synthesis of N(tEB)₂2 and its maleimide derivative, N(tEB)₂ 2-mal, followed the synthetic scheme in FIG. 5.

To a 20 mL glass vial containing 100.0 mg of o-tolidine and 200.0 mg of NOTA-3HCl in 4 mL of dimethyl Sulfoxide (DMSO) was added 100 μL diisopropylethylamine and 90 μL diethyl cyanophosphonate. The mixture was stirred at room temperature overnight. The mixture was then purified with semi-preparative HPLC. The peak containing the desired product was collected and the solution was frozen over dry ice and lyophilized overnight to give 28.5 mg pure tolidine-NOTA-tolidine (compound 13). To a 20 ml glass vial containing 12.7 mg of tolidine-NOTA-tolidine in 1.0 mL of water was added 110 μmole of HCl (11 μL). The mixture was cooled in ice bath and 7.6 mg of sodium nitrite (110 μmol) in 0.5 mL of water was added to the vial drop wise. The mixture was stirred in ice bath for 20 min and the yellow diazonium salt solution (compound 14) was added drop wise to another vial in ice bath containing 20.0 mg of 1-amino-8-naphthol-2,4-disulfonic acid and 13.0 mg of sodium bicarbonate in 0.5 mL of water. The mixture was stirred in ice bath for 3 hours and purified with semi-preparative HPLC. The product was collected and lyophilized overnight to give 15.0 mg pure NOTA-N(tEB)₂ (compound 15; also designated “N(tEB)₂ 2” herein.). To a 20 mL glass vial containing 12.0 mg of NOTA-N(tEB)₂ and 11 mg of N-(2-aminoethyl)maleimide in 4 mL of DMF was added 20 mg of HATU and 50 μL DIPEA. The mixture was stirred at room temperature for 2 hours and purified with HPLC to give 8.5 mg of maleimide-labeled NOTA-N(tEB)₂ (“N(tEB)₂ 2-mal”) after lyophilization.

### Example 4: Characterization of Complexes of NtEB and N(tEB)₂ with has

In all of the following experiments, N(tEB)2 means the N(tEB)2 2 compound.

Morphology and Size

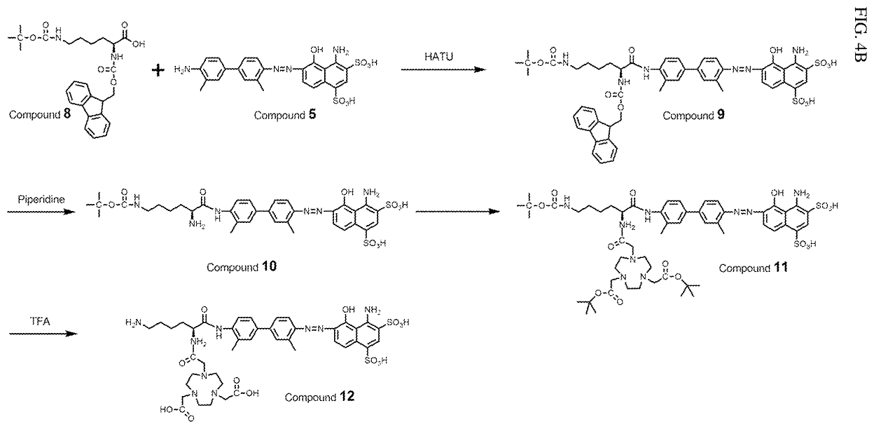

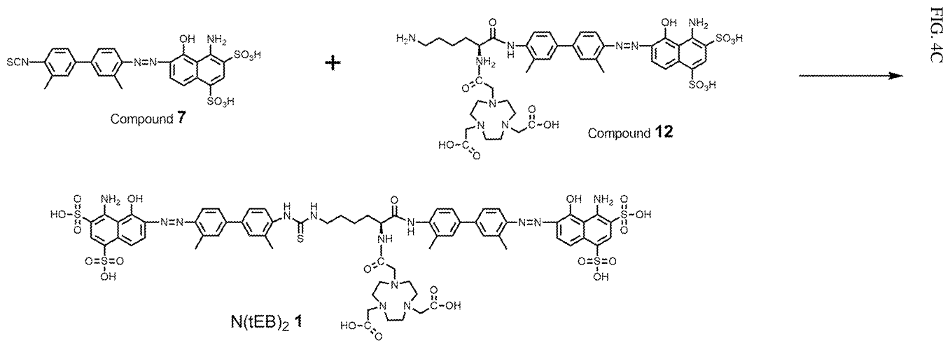

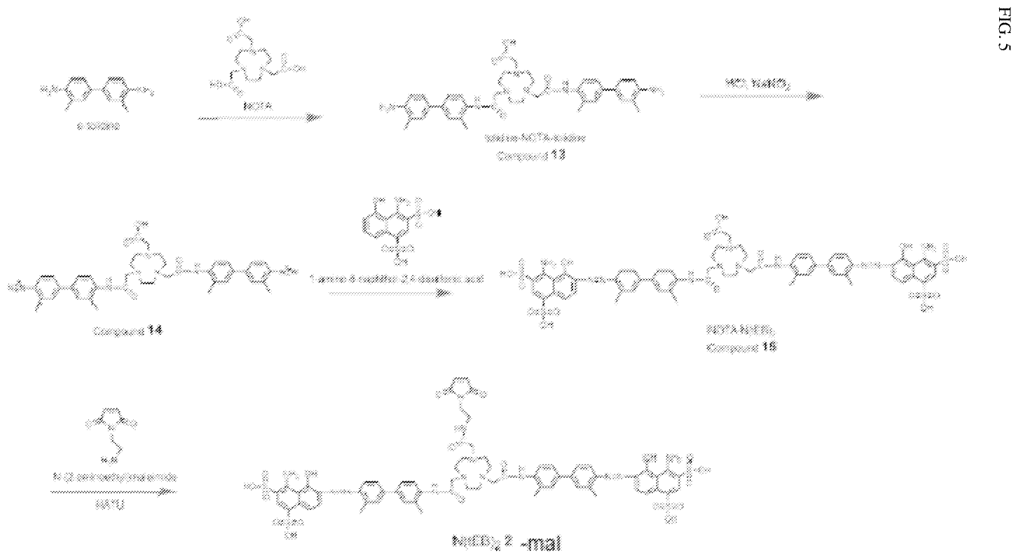

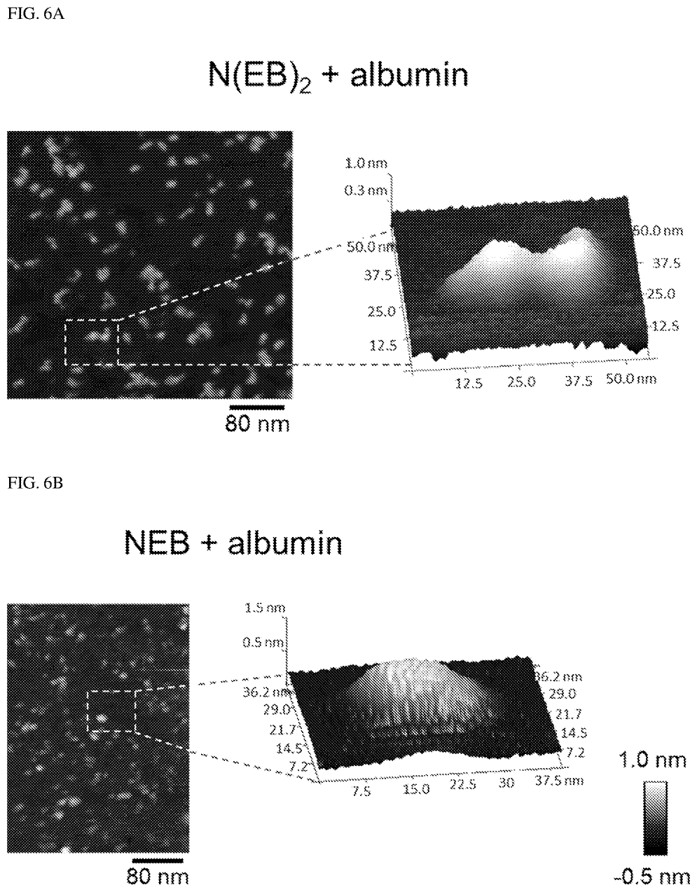

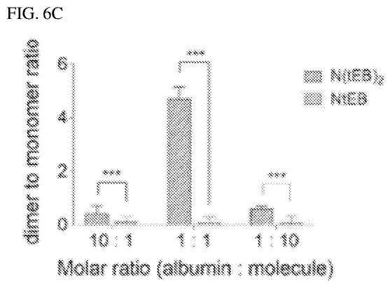

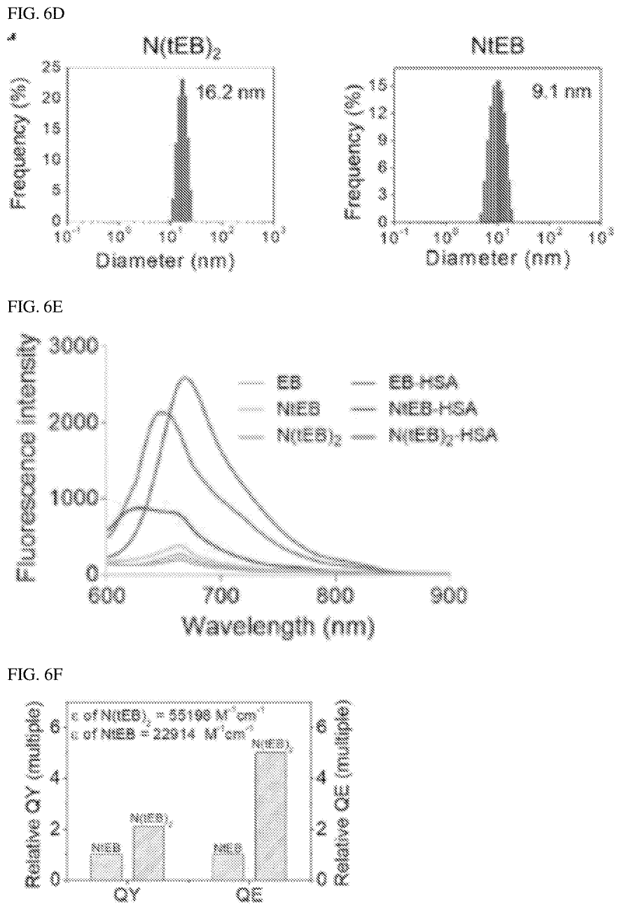

We performed atomic force microscopy (AFM) to study the morphological structures of the complex. NtEB and N(tEB)₂ were respectively incubated with human serum albumin (HSA) (molar ratio of 1:10, 1:1 and 10: 1) at room temperature for 30 min. Samples (10 L) were cast on freshly peeled mica substrate, followed by drying, rinsing, and dehumidifying. AFM was carried out in tapping mode in air on a PicoForce Multimode AFM (Bruker, CA) equipped with a NANOSCOPE V controller, a type E scanner head, and a sharpened TESP-SS (Bruker, CA) AFM cantilever. An inverted optical microscope (IX71, Olympus, Japan) was used to capture pictures. AFM images were then analyzed by NANOSCOPE Software (version 7.3-8.15, Bruker, CA). For quantification, 10 different fields of view in one image were selected to quantify the “dimer” and the “monomer”. The size and morphology of dimer and monomer was defined by ImageJ (NIH, MD), a public domain Java image processing program. The dimer was found to be dumbbell-shaped and have a length: >25 nm, while the monomer morphology was not dumbbell-shaped and had length ≤25 nm and width ≤25 nm. (FIG. 6A,B) Dimerization of albumin mediated by N(tEB)₂ was observed at all tested molar ratios, while no apparent albumin dimers was identified in the mixture of NtEB and HSA (FIG. 6C).

These results were further substantiated by transmission electron microscopy (TEM), dynamic light scattering (DLS) and high-resolution LC-MS.

TEM: Human serum albumin (HSA) (1 mg/mL) was mixed with NtEB or N(tEB)₂ at a molar ratio of 1:1, respectively for characterization by TEM. The TEM samples were prepared by depositing a drop of the solution (1 mg/mL HSA) on the surface of a copper net coated with carbon. Images were obtained using a Philips/FEI CM200 Microscope (USA). Every protein sample was imaged for at least three times independently and each sample was observed in more than five regions to avoid experimental errors. The size of the N(tEB)₂-albumin dimer determined by TEM was 17.1±3.2 nm, significantly larger than that of NtEB-albumin monomer (9.3±0.6 nm).

Dynamic light scattering (DLS) obtains a hydrodynamic diameter based on the diffusion of the particles. DLS was performed as follows. In the samples of NtEB-HSA and N(tEB)₂—HSA dimer, the concentration of HSA was 1 mg/mL and the molar ratio of HSA to NtEB or N(tEB)₂ was 1:1. The hydrodynamic diameter of each of NtEB-HSA, N(tEB)₂—HSA dimer, and HSA was measured using DLS SZ-100 Nanoparticle Analyzer (HORIBA Scientific, Japan), respectively. The mean value of triplicate measurements was used for analysis.

The hydrodynamic diameters determined in solution by DLS were 6.7, 9.1, and 16.2 nm for free albumin, NtEB-albumin, and N(tEB)₂-albumin₂, respectively (FIG. 6D).

N(tEB)₂ Shows Increased HSA Binding Affinity and Fluorescence Efficiency

Binding affinity with albumin of each of EB, NtEB, N(tEB)₂, NtEB-exendin-4, and N(tEB)₂-exendin-4 was determined by biolayer interferometry (BLI) using biotinylated human serum albumin (HSA)/streptavidin biosensors using an OCTET Red96 system (FortdBio, LLC). In this study, we used the streptavidin conjugated biosensors (Pall Fort6Bio LLC, CA). We preincubated the biosensors with biotinylated HSA (Abeam Inc., Cambridge, Mass.) for 10 minutes. After removing the unbound biotinylated HSA, the biosensors were washed for 1 minute. After blocking the spare biosensor, we added EZ-Link™ Sulfo-NHS-Biotin (Thermo Fisher Scientific, Rockford, Ill.) to each well and then removed it and washed the wells. We then added each sample (NtEB or N(tEB)2) to the wells.

A dilution series of each compound (100, 50, 25, 12.5, 6.25, 3.125, 1.526 μM) in 1× phosphate buffered saline (PBS), pH 7.4, was used to delineate the binding profile with the albumin. Association of a given dilution of a compound with the albumin-derivatized biosensor was measured for 600 seconds and then in turn, dissociation of the compound from the albumin measured for another 600 seconds. Data was analyzed using the OCTET Data Analysis software 7.0. The results for binding of EB, NtEB, N(tEB)₂ with HSA are tabulated below in Table 1.

TABLE 1
Kd, Kon and Koff for EB dye, NtEB and N(tEB)2

| | Goodness of fit | Kₒₙ | K_{off} | K_{d} |
|---|---|---|---|---|
| Name | (R²) | (1/μM · s) | (1/s) | (μM) |
| EB | 0.9590 | 4.5094 × 10⁴ | 0.1778 | 3.7 |
| NtEB | 0.9986 | 1.3451 × 10⁴ | 0.7947 | 79 |
| N(tEB)₂ | 0.9790 | 4.7128 × l0⁴ | 0.1009 | 1.8 |

The K_{d} value of the N(tEB)₂ complex with the albumin (K_{d}=1.8 μM) is 43 times lower than the K_{d} value of the NtEB complex with albumin (K_{d}=79 μM) and 2 times lower than the K_{d} value of the EB complex with albumin (K_{d}=3.7 μM). The albumin complex with N(tEB)₂ additionally showed a relatively high Kₒₙ value (4.71×10⁴ M⁻¹ s⁻¹), and low k_{off} value (0.10 s⁻¹), compared to the kinetics for the albumin complexes with EB and NtEB (FIG. 6E and Table 1 above). The quick association and slow dissociation with albumin further governed the favorable binding capacity of N(tEB)₂.

The absorption and the emission spectra of N(tEB)₂, NtEB and EB were measured in PBS, BSA and FBS buffers. The absorption peaks of N(tEB)₂, NtEB and EB in BSA were located at 610, 550, and 548 nm, respectively. The similar emission peaks of the three EB derivatives in BSA were recorded at 661, 652 and 660 nm, respectively.

Fluorescence brightness of each compound was further compared with equal molar amount of N(tEB)₂, NtEB, or EB in the presence or absence of human serum albumin. The enhanced fluorescence intensity of N(tEB)₂-albumin dimer relative to N(tEB)₂ alone was comparable with that of EB-albumin relative to EB alone, while the signal intensity was over two times higher than that of the NtEB-albumin complex (FIG. 6E).

Quantum efficiency is a dye metric encompassing both the quantum yield and absorption coefficient (QE=ε*QY). A detailed quantum efficiency analysis revealed that N(tEB)₂ yielded a 2-fold quantum yield enhancement and 5-fold QE improvement over NtEB based on the enhanced extinction coefficient of N(tEB)₂ (55198 M⁻¹ cm⁻¹) over NtEB (22914 M⁻¹ cm⁻¹) (FIG. 6F). We observed fluorescence enhancement of N(tEB)₂ when switching to a BSA/fetal bovine serum (FBS) buffers, as shown in Table 2 below.

TABLE 2
Optical properties of EB dye, NtEB, and N(tEB)₂

| | Sample | MW (g/mol) | OD₆₀₀ₙₘ | ODₚₑₐₖ | ϵ₆₀₀ₙₘ | ϵₚₑₐₖ | Molar ϵ₆₀₀ₙₘ | Molar ϵₚₑₐₖ | QY₆₀₀ₙₘ | QE |
|---|---|---|---|---|---|---|---|---|---|---|
| In PBS | NtEB | 827.23 | 0.0857 | 0.1533 | 21.43 | 38.33 | 17723.31 | 31703.42 | — | — |
| | N(tEB)₂ | 1351.31 | 0.0987 | 0.1806 | 22.03 | 40.31 | 29771.00 | 54474.60 | — | — |
| | EB | 960.81 | 0.9906 | 0.9983 | 96.47 | 97.22 | 92693.55 | 93414.07 | — | — |
| In BSA | NtEB | 827.21 | 0.0759 | 0.1108 | 18.98 | 27.70 | 15696.60 | 22914.15 | 1 | 15696.60 |
| | N(tEB)₂ | 1351.31 | 0.1245 | 0.183 | 27.79 | 40.85 | 37553.09 | 55198.51 | 2.10 | 78929.01 |
| | EB | 960.81 | 0.905 | 0.9551 | 88.14 | 93.02 | 84683.69 | 89371.71 | — | — |
| In FBS | NtEB | 827.23 | 0.0883 | 0.1132 | 22.08 | 28.30 | 18261.00 | 23410.48 | 1 | 18261.00 |
| | N(tEB)₂ | 1351.31 | 0.1561 | 0.1931 | 34.84 | 43.10 | 47084.63 | 58244.99 | 2.10 | 98962.49 |
| | EB | 960.81 | 0.8414 | 0.9057 | 81.94 | 88.21 | 78732.44 | 84749.19 | — | — |

This fluorescence enhancement in the BSA and FBS buffers indicates that N(tEB)₂ can show significant fluorescence as a result of its in vivo albumin binding.

### Example 5: N(tEB)₂ Shows Improved Pharmacokinetics and Enhanced Tumor Accumulation

In vivo dynamic positron emission tomography (PET) was performed in healthy mice to determine the pharmacokinetics of ¹⁸F labeled N(tEB)₂ or NtEB.

Preparation of ¹⁸F, ⁶⁴Cu Labeled NtEB, N(tEB)₂

Radionuclides ¹⁸F and ⁶⁴Cu were produced and supplied by the Clinical Center's cyclotron facility of the National Institute of Health (NIH). The method and procedure for preparation of ¹⁸F or ⁶⁴Cu labeled N(tEB)₂ was according to the NtEB labeling procedure reported in Niu, et al. In Vivo Labeling of Serum Albumin for PET. J. Nucl. Med. 55, 1150-1156 (2014).

Specifically, ⁶⁴CuCl₂ was converted to ⁶⁴Cu-acetate by adding 0.5 mL of 0.4 M NH₄OAc solution (pH 5.6) to 20 μL ⁶⁴CuCl₂. ⁶⁴Cu-acetate solution (0.1 mL; 3-4 mCi) was added into a solution of 100 μg of NtEB or N(tEB)₂ in water (10 mg/mL). The mixtures were put on the orbital shaker (250 rpm) for 0.5 h at 37° C. Then, the radiochemical purity was determined using iTLC plates (Fisher Scientific), developed in 0.1M citric acid (pH 5). NtEB and N(tEB)₂ were purified by a C18 Sep-Pak (BOND-ELUT 100 mg, Varian), followed by eluted from the cartridge using 70% ethanol and 30% PBS.

To prepare ¹⁸F-AlF-labeled NtEB or N(tEB)₂, 0.13 mL acetonitrile and 0.05 mL aqueous ¹⁸F-fluoride (0.3-0.9 GBq) was added to a 1-mL plastic tube containing 3 mL 2 mM aluminum chloride in 0.5 M, pH 4.0, sodium acetate buffer and 6 mL of 3 mM NtEB or N(tEB)₂ in 0.5 M, pH 4.0, sodium acetate buffer. The mixture was stirred in a vortex mixer and heated in a 105° C. heating block for 10 min. The vial was cooled, and the solution was diluted with 10 mL of water and trapped on a Varian Bond Elut C18 column (100 mg). The radioactivity trapped on the C18 column was eluted with 0.3 mL of 80% ethanol/water containing 1 mM HCl. The ethanol solution was evaporated with argon flow, and the final product was dissolved in phosphate-buffered saline and analyzed by HPLC.

PET Imaging

All PET images were acquired using an Inveon PET scanner (Siemens Preclinical Solutions, Malvern, Pa.). Mice were anesthetized using isoflurane/O₂ (2% v/v) before injection. The images were reconstructed using a 2-dimensional ordered-subset expectation maximum algorithm, and no correction was applied for attenuation or scatter. For each scan, regions of interest (ROIs) were drawn using vendor software (ASI Pro 5.2.4.0; Siemens Medical Solutions) on decay-corrected whole-body coronal images. The radioactivity concentrations (accumulation) within the heart, muscle, liver, and kidneys were obtained from mean pixel values within the multiple ROI volumes and then converted to megabecquerel per milliliter. These values were then divided by the administered activity to obtain (assuming a tissue density of 1 g/mL) an image-ROI-derived percentage injected dose per gram (% ID/g).

Comparison of the half-life of the N(tEB)₂ and NtEB was performed by two-phase linear regression of the time activity curves over heart at various time points, respectively. The analysis was performed as in Yu, M. & Zheng, J. Clearance pathways and tumor targeting of imaging nanoparticles. ACS Nano. 9, 6655-6674 (2015), and the data was calculated using GraphPad Prism 7.03 (GraphPad Software Inc., San Diego, Calif.).

The mice were killed at specified time points. Organs and blood were collected and wet weighted. The collected organs and blood, together with a series of standard solutions, were measured for ⁶⁴Cu radioactivity on a gamma counter (Wallac Wizard 1480, PerkinElmer). The radioactivity of organs and blood was converted to calculate the percentages of the injected dose (% ID) in organs of interest and the percentages of the injected dose per gram of tissue (% ID/g).

In Vivo Pharmacokinetics

For the in vivo pharmacokinetics study, healthy BALB/c mice (5 mice in each group) were administered 3.7 MBq (100 μCi) ¹⁸F-labeled NtEB or N(tEB)₂ via tail vein injection and 60-min dynamic PET acquisitions were performed.

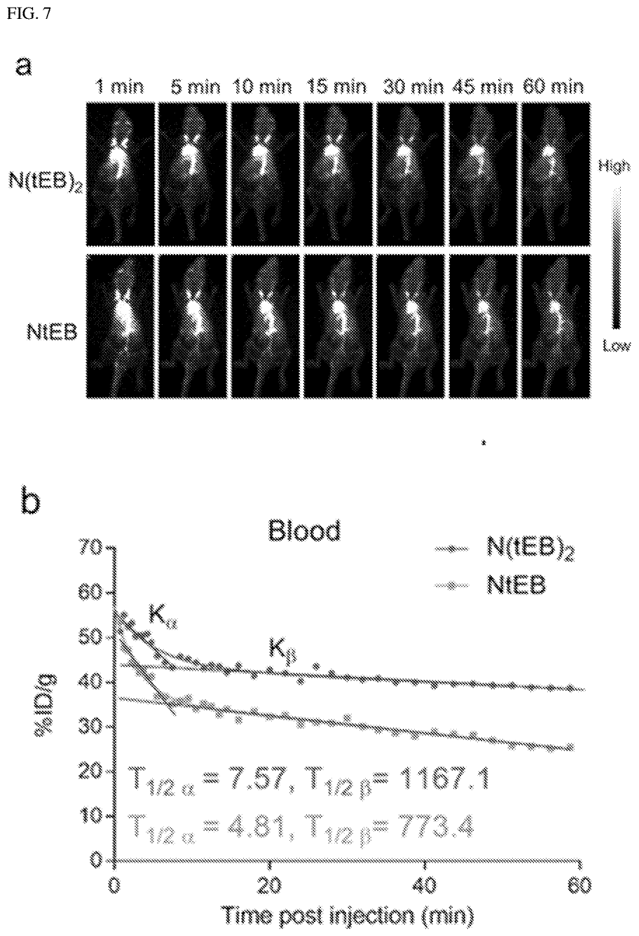

After intravenous administration, both ¹⁸F—N(tEB)₂ and ¹⁸F-NtEB showed high radioactivity accumulation and retention in the circulatory system, with clear delineation of highly perfused organs including heart, liver, kidneys and spleen (FIG. 7a). Regions of interest (ROIs) were drawn over different organs to generate time-activity curves (TACs). Based on the TACs, linear regression was used to estimate the dominant half-life and clearance of these two tracers. As expected, ¹⁸F—N(tEB)₂ showed 1.66 times slower clearance from circulation in vivo than ¹⁸F-NtEB (FIG. 7b), indicating the potential of ¹⁸F—N(tEB)₂ to be used as a blood pool imaging agent.

Tumor Retention of N(tEB)₂

Due to the aberrant and leaky vasculature within tumor tissues, drug delivery based on enhanced permeability and retention (EPR) effect has been a well-established strategy. Long circulation half-life is the prerequisite for EPR-based drug delivery. Tumor retention of N(tEB)₂ was assessed with PET imaging in several tumor mouse xenograft models with different levels of blood supply and vascular permeability, after labeling with ⁶⁴Cu (t_{1/2}=12.6 h).

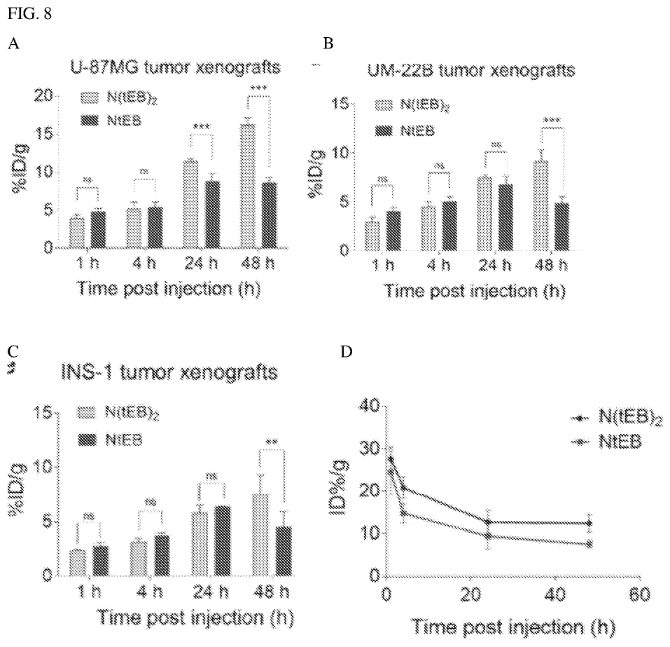

For the tumor uptake study, PET scans were performed at 22-28 days post inoculation when the tumor volume reached about 200-300 mm³. 3.7 MBq ⁶⁴Cu labeled NtEB or N(tEB)₂ were injected to nude mice (5-6 mice in per group) via tail vein and PET images were acquired 4 h, 24 h and 48 h post-injection (p.i.) with ⁶⁴Cu labeled NtEB or N(tEB)₂. PET images were reconstructed without correction for attenuation or scattering using a three-dimensional ordered subsets expectation maximization algorithm. ASI Pro VMTM software was used for image analysis. Regions of interest (ROI) were drawn on LNs to calculate the % ID/g. Results of the quantification for each of the three tumor models as a function of time are shown in FIG. 8c-e.

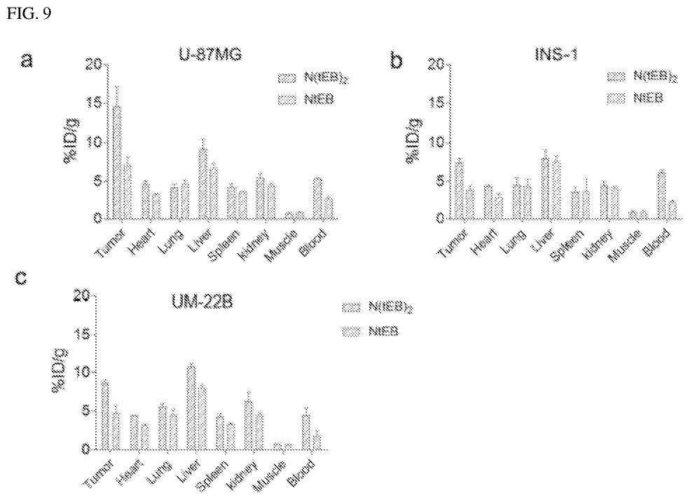

Compared with NtEB, the tumor uptake of N(tEB)₂ was significantly improved in all tested tumor models at late time points (24 and/or 48 h post-injection (p.i.)), despite UM-22B and INS-1 exhibiting a relatively slower maximal accumulation than U-87MG (FIG. 8A-C). Additionally, the clearance of ⁶⁴Cu—N(tEB)₂ was slower than the ⁶⁴Cu-NtEB in the blood pool, which contributes to the higher retention in tumor (FIG. 8D). The high retention of N(tEB)₂ in tumor sites was further corroborated with the ex vivo bio-distributions study at 48 h p.i. (FIG. 9). The ⁶⁴Cu—N(tEB)₂ showed higher accumulation in tumor than that of ⁶⁴Cu-NtEB in all three tumor bearing mice models. As shown in FIG. 9, the four organs showing the highest uptake of ⁶⁴Cu—N(tEB)₂ at 48 h p.i. were tumor, liver, blood/heart, kidney.

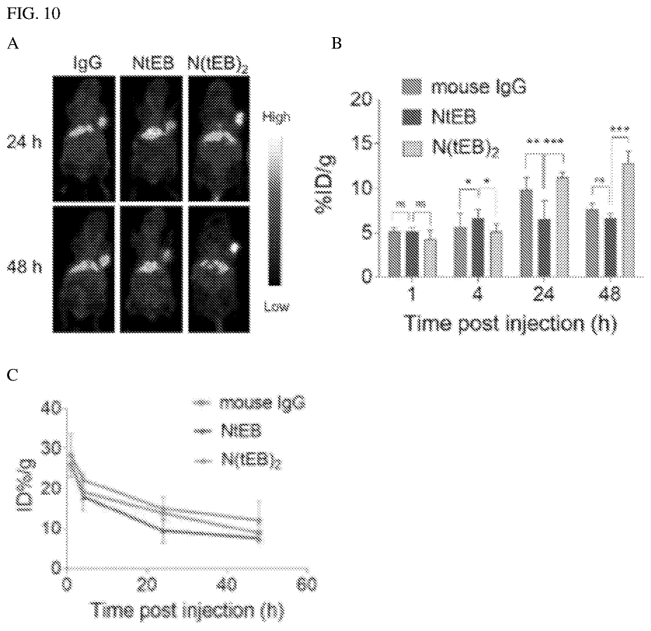

The overall size of N(tEB)₂-albumin dimer is more than 130 kDa, which is similar to immunoglobulin G (IgG) in regard to molecular weight and hydrodynamic diameter. PET imaging revealed comparable tumor retention of N(tEB)₂ and IgG at 24 h p.i. However, the tumor retention of N(tEB)₂ was significantly higher than that of IgG at 48 h p.i., indicating the former is more efficient for EPR mediated tumor delivery (FIG. 10A-C).

### Example 6: In Vivo Albumin Dimer Selectively Mapped the Sentinel Lymph Nodes

The existence of albumin in the interstitial fluid within the lymphatic system makes EB derivatives ideal for lymphatic mapping.

For the lymph node mapping study, 0.37 MBq/¹⁸F labeled NtEB or N(tEB)₂ in 10 L saline was injected into the footpad of the mice (5 mice in each group) (Siemens Medical Solutions, Malvern, Pa.). 60-min dynamic PET acquisitions were performed, and additional static PET images were acquired at 90 min and 120 min p.i. Then, the radiolabeled NtEB or N(tEB)₂ were simultaneously injected into the contralateral foot pads of the same mice to rule out the influence of individual variance.

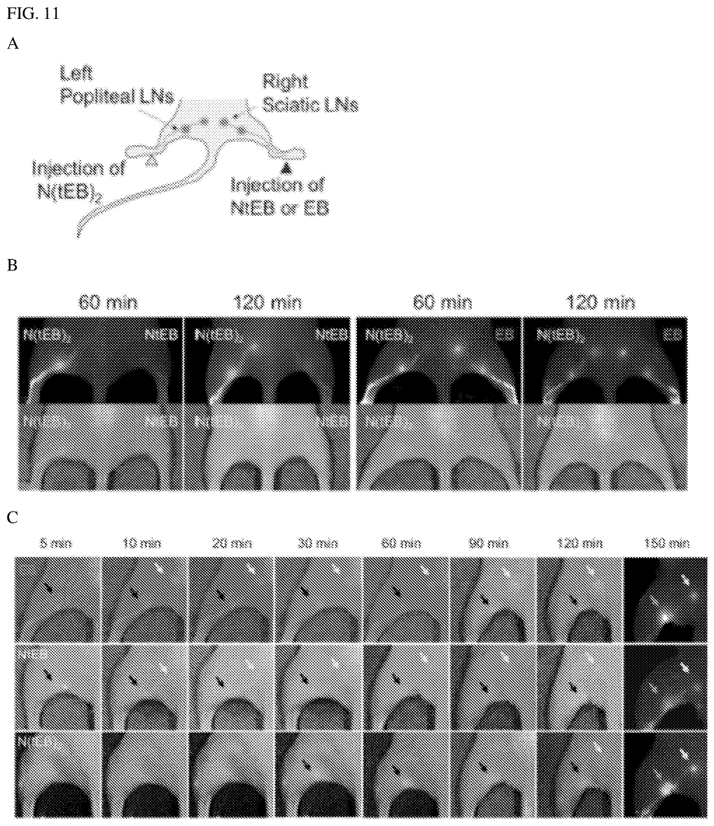

By binding albumin after local injection, N(tEB)₂ was able to overcome several shortcomings of NtEB in sentinel lymph node biopsy (SLNB). The fluorescence yield of N(tEB)₂ was superior to NtEB. After equivalent 10 μg dosages of N(tEB)₂ or NtEB were simultaneously injected into the contralateral foot pads of normal mice, the high fluorescence intensity of N(tEB)₂ helped distinguish lymphatic vessels and associated lymph nodes (LNs) (FIG. 11A, B). Although N(tEB)₂ produced nearly identical LN imaging quality compared with EB dye, EB illuminated the sentinel and secondary LNs within 10 min and it cannot be modified (FIG. 11B). More importantly, for successful SLNB, the time window between visualization of sentinel lymph nodes and illumination of secondary lymph nodes is critical to ensure only tumor draining lymph nodes are excised for intraoperative pathologic examination. In the mouse model, the time window between detection of the popliteal LN and the sciatic LN was around 10 minutes post administration of NtEB or EB (FIG. 11C). In contrast, the popliteal LN was visualized at 30 min p.i. of N(tEB)₂ while the sciatic LN were illuminated at around 90 min p.i., producing a more operable time window of up to 60 min for imaging-guided SLNB.

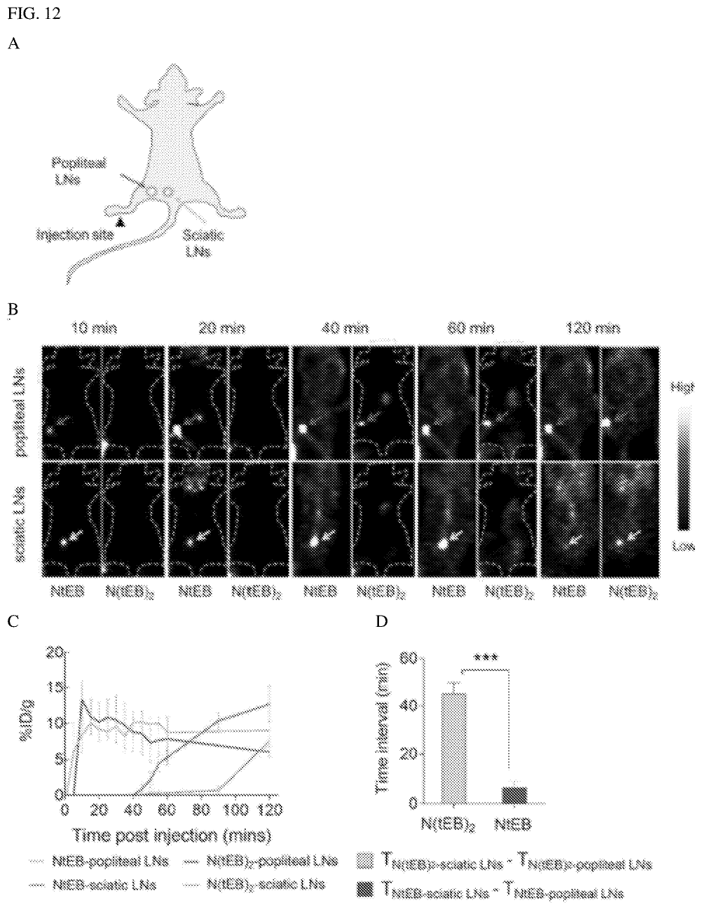

Compared to fluorescence optical imaging, PET offers deeper tissue penetration and higher sensitivity. Both popliteal and sciatic LNs were clearly visualized with high contrast on PET images using ¹⁸F-labeled N(tEB)₂ or NtEB as the imaging probe (FIG. 12A,B). Similar to the results from optical imaging, a time window of approximately 50 min between sentinel LNs (popliteal) and secondary LNs (sciatic) detection was observed from PET imaging (FIG. 12C,D). To rule out the influence of individual variance, NtEB and N(tEB)₂ were injected in the same mouse on different foot pads, and the results were consistent for the visualization of LNs and time window between primary and secondary LNs. Collectively, the high binding affinity of N(tEB)₂ to albumin, the increased fluorescence brightness and slow migration of N(tEB)₂ crosslinked albumin dimer in lymphatic system make N(tEB)₂ an ideal imaging probe for SLNB using either optical or PET imaging. Moreover, the purple color of N(tEB)₂ in bright field enables trimodal imaging to further improve diagnostic accuracy for informed decision making and surgical guidance.

### Example 7: N(tEB)₂ Protects Drug from Enzymatic Degradation

It was hypothesized that when a peptide is attached to a N(tEB)₂ through a coupling reaction, for example a thiol-maleimide reaction, the peptide will be protected from enzymatic degradation while it in complex with albumin dimers via N(tEB)₂ by being sandwiched between the two albumins.

To test the hypothesis, exendin-4 peptide, a glucagon-like peptide-1 (GLP-1) agonist, was linked to N(tEB)₂ and NtEB to provide two conjugates, N(tEB)₂-exendin-4 and NtEB-exendin-4, respectively.

### Synthesis of N(tEB)₂-exendin-4

The N(tEB)₂-exendin-4 conjugate was obtained by mixing 2.26 mg of cys-40-exendin-4 and 0.78 mg of maleimide-N(tEB)₂ in 1 mL of water. The LC-MS analysis showed the formation of desired product.

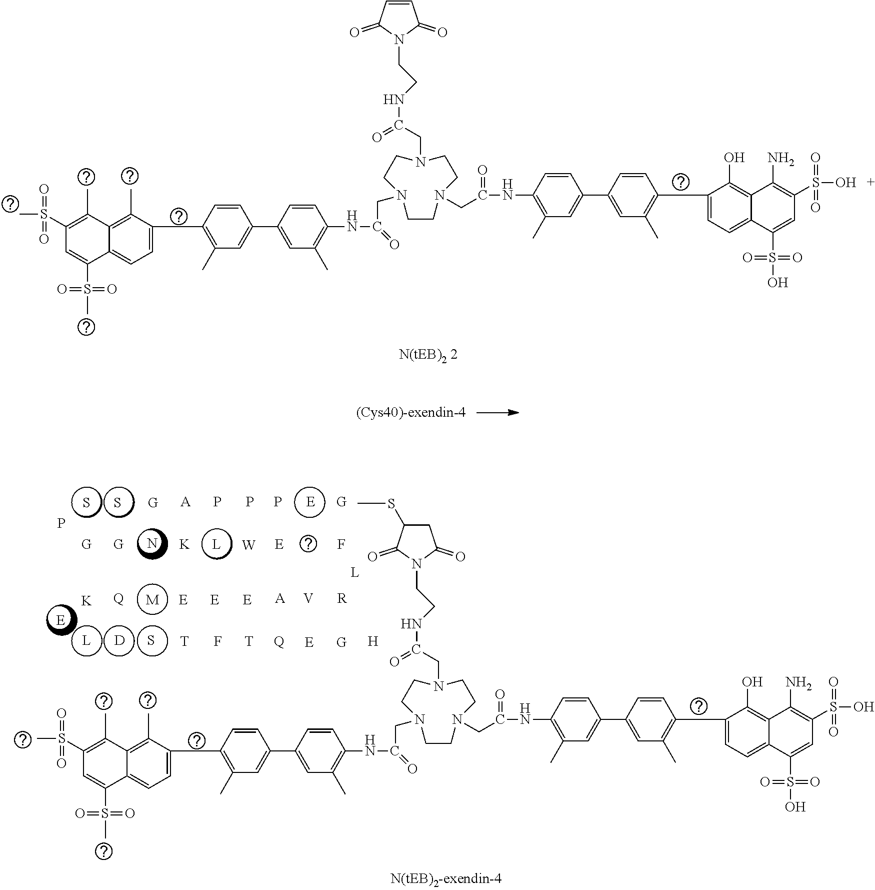

### Synthesis of EB-Exendin-4

To a solution of cys-40-Exendin-4 (6.3 mg) in 3 mL PBS buffer (pH 7.0) was added 2.0 mg of maleimide-EB (compound Ia in the scheme below). The mixture was stirred at room temperature and monitored with HPLC. After the completion of the reaction, the mixture was purified with semi-prep HPLC in 5 injections. The fractions containing the product were collected and lyophilized to give 7.2 mg of desired product, EB-Exendin-4. LC-MS: [MH]⁺=4911.00, calc: 4912.32.

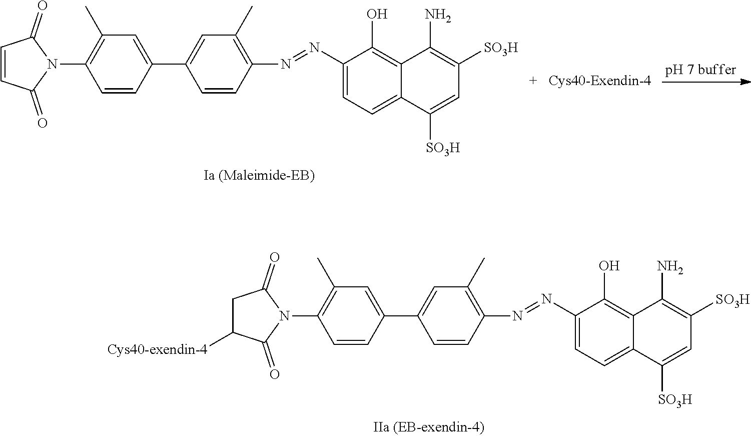

Binding Affinity for Human Serum Albumin

Affinity for human serum albumin and the kinetics of binding of N(tEB)₂-exendin-4 and NtEB-exendin-4 were measured by bio-interferometry at concentrations ranging from 1.56 to 100 μM for NtEB-exendin-4 and 3.125 to 100 μM for N(tEB)₂-exendin-4. The computed Kd, Kon, and Koff values are shown in the Table 3 below.

TABLE 3
Ka, Kₒₙ and K_{off} for NtEB-exendin-4 and N(tEB)₂-exendin-4

| | Goodness of fit | K_{d} | Kₒₙ | K_{off} |
|---|---|---|---|---|
| Compound | (R²) | (μM) | (1/μM · s) | (1/s) |
| N(tEB)₂-exendin-4 | 0.9871 | 0.68 | 2818916 | 0.04626 |
| NtEB-exendin-4 | 0.9359 | 1.4 | 3340766 | 0.06249 |

N(tEB)₂-exendin-4 showed significant higher binding affinity with albumin than NtEB-exendin-4 (K_{d}˜ 0.68 μM vs. 1.4 μM), with relatively fast association and slow dissociation.

Proteolysis

A trypsin digestion study was performed with N(tEB)₂-exendin-4 to investigate the peptide protection ability of the N(tEB)₂-albumin dimer complex. The exendin-4 peptide contains one arginine and two lysine residues which are cleavage sites for trypsin.

For the enzymatic degradation setup, 80 μL exendin-4 (0.5 mg/mL) was incubated with trypsin (0.05 mg/mL) for different time (0, 5, 20, and 40 min) at 37° C. on orbital shaker (250 rpm). Before subjection to trypsin, the freshly prepared mixture of CH₃CN (75%) and formic acid (4%) was used as the stop solution for enzymatic reaction. The whole reaction solution was analyzed and most dominant fragments were assigned to specific molecules.

For evaluating the anti-degradation effect resulting from binding to albumin, 80 μL free exendin-4, NtEB-exendin-4, and N(tEB)₂-exendin-4 were preincubated with HSA (20 mg/ml) for 30 min at 37° C. on the orbital shaker (250 rpm). The molar ratio of compound to HSA was optimized to 1:5. Before subjection to trypsin, the freshly prepared mixture of CH₃CN (75%) and formic acid (4%) was used as the stop solution for enzymatic reaction. The samples were subjected to trypsin (optimized to 0.05 mg/mL) digestion. 30 μL sample at various time points (0, 5, 10, 20, 30, 50 min post treatment) was transferred into a 1.5 mL tube, and then 30 μL stop solution was immediately added to each tube to stop reaction. The samples (60 μL in total) were put in a dry ice box. Before LC/MS, all the samples were thawed to room temperature.

For quantitative analysis of fragments from the enzymatic reaction, the LC/MS system consisted of an Agilent 1200 autosampler, Agilent 1200 LC pump, and an AB/MDS Sciex 4000 Q TRAP (Life Technologies Corporation, Carlsbad, Calif.). Separation was achieved on an Phenomenex Gemini column (5 m, 110 A, 50 mm×4.6 mm) with 2 mM ammonium acetate and CH₃CN with the following gradient system at a flow rate of 1.0 mL/min: 100% (v/v) A and 0% B for 1 min initially, gradient from 0 to 46% B over 4 min, isocratic elution at 46% B for an additional 5 min, washing with 100% B over 1 min, and re-equilibrium with A for an additional 1 min. Different combinations of multiple-reaction monitoring (MRM) and full scan MS/MS experiments were performed. Three replicate injections (10 μL) were made for each time-point metabolite. The specific comparisons made for quantitation used a single MRM transition per analyte.

When exendin-4 was subjected to trypsin digestion, four main fragments were observed using LC-MS at various incubation times, as expected

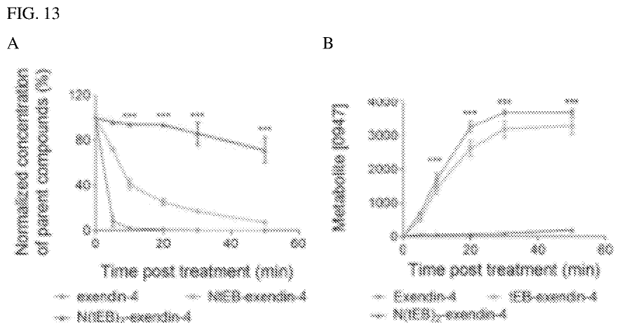

Remarkably, N(tEB)₂-exendin-4 showed highest resistance to trypsin degradation in the presence of albumin, with ˜70% of the exendin-4 intact after incubating with trypsin for 50 min, which is significantly higher than that of N(tEB) exendin-4 (˜10%, P<0.001) and exendin-4 (<0.1%, P<0.001) (FIG. 13A).

From the four major fragments of exendin-4, we chose one fragment commonly shared by N(tEB)₂-exendin-4, NtEB-exendin-4 and exendin-4 for further quantification. The fragments generated from N(tEB)₂-exendin-4 consistently increased much slower than that from NtEB-exendin-4 and exendin-4, further confirming peptide protection effect of N(tEB)₂ through sandwiching the peptide by albumin proteins (FIG. 13B).

### Example 8: N(tEB)₂-Exendin-4 Shows Enhanced Antidiabetic Efficacy

Based on the structure of the N(tEB)₂-albumin dimer and its favorable in vivo behavior, it was hypothesized that a peptide conjugated to N(tEB)₂ will be endowed with extended circulation half-life and consequently, enhanced therapeutic efficacy.

In this study, we used db/db mice (6-8 weeks, male, 40-50 g; Harlan Laboratories). These mice received a single subcutaneous injection of exendin-4, NtEB-exendin-4, N(tEB)₂-exendin-4, or semaglutide (30 nmol/kg body weight, n=3/group), respectively. The plasma-equivalent glucose was measured from tail vein blood samples (˜5 μl) of mice using a True-Track glucose meter (CVS Health, USA). For evaluation of pharmacokinetics, the concentration of exendin-4 was measured using ELISA in venous blood samples acquired at multiple time points post subcutaneous injection. Plasma Exendin-4 levels were determined by a commercial Exendin-4 ELISA kit (Phoenix Biotech, USA) according to the manufacturer's instructions. Briefly, 25 μL blood samples collected at different time points from the mice were added to the microwells of the plate, after incubation and washing, 100 μL SA-HRP was added to each well and incubated them for 1 hour. The solution was removed and washed, followed by TMB substrate solution adding to each well. Then, 100 μL HCl was added to stop the reaction. The results were observed by a Microplate Reader.

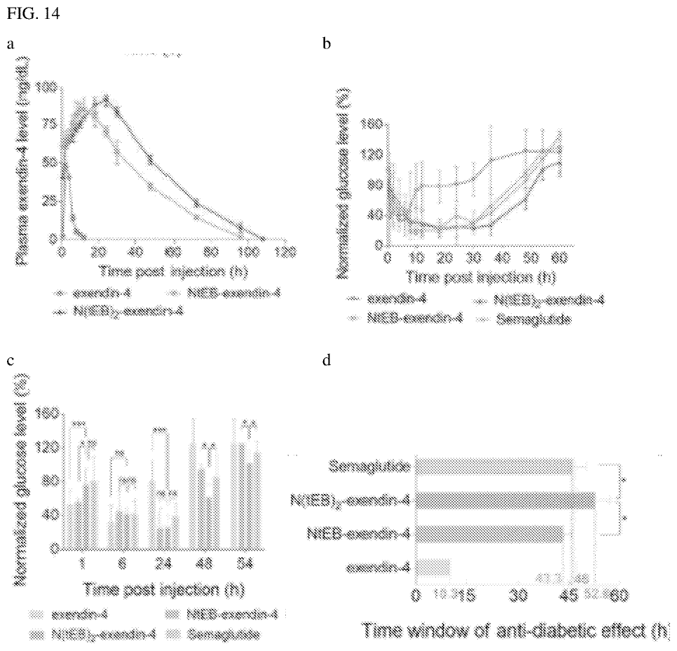

Exendin-4 alone showed fast entry into circulation from injection site, and cleared from the body within 12 h p.i. Compared with free exendin-4, NtEB-exendin-4 showed dramatic increase in circulation time with the peak concentration observed at 12 h p.i., and clearance by 96 h p.i. Lastly, N(tEB)₂-exendin-4 also exhibited significantly prolonged release of exendin-4 with peak concentration of exendin-4 at 24 h p.i., and retention time up to 108 h p.i. (FIG. 14a).

The hypoglycemic properties of free exendin-4, NtEB-exendin-4 and N(tEB)₂-exendin-4 were tested in type 2 diabetes mellitus (T2DM) mice after subcutaneous injection. Semaglutide, a long-acting GLP-1 agonist which recently received FDA approval and arguably the best commercial weekly formula so far, was used as the positive control.

Hypoglycemic efficacy of N(tEB)₂-exendin-4 was evaluated using a glucose tolerance test in male db/db mice (6-8 weeks). Saline, exendin-4, NtEB-exendin-4 and a commercially available hypoglycemic drug semaglutide were also tested. Under non-fasting conditions with free access to food and water, animals were administrated with a single dose of subcutaneous injection of saline, exendin-4, semaglutide, NtEB-exendin-4 or N(tEB)₂-exendin-4 (25 nmol/kg body weight, n=3/group). Blood samples were collected from tail vein at different time points (0, 15, 30, 60, 90, 120 mins) post administration, and blood glucose levels were monitored with a blood glucose meter (ACCU-CHEK Sensor, Roche Diagnostics Corp., USA)

The glucose levels of the four treated cohorts were monitored at different time points post administration. After baseline plasma glucose concentration normalization, it was observed that glucose level was reduced by approximately 50% at 1 h p.i. of free exendin-4 and NtEB-exendin-4, and by approximately 20% at 1 h p.i. of N(tEB)₂-exendin-4 and semaglutide. This was attributed to the delayed release and enhanced residence time of N(tEB)₂-exendin-4 and semaglutide (FIG. 14b,c). The glucose recovery time of NtEB-exendin-4 (515.00±25.0 mg/dL at 48 h), semaglutide (475.33±55.4 mg/dL at 54 h) and N(tEB)₂-exendin-4 (519±5.35 mg/dL at 54 h) treated mice were much longer than free exendin-4 (389.67±44.3 mg/dL at 12 h). The effective time window of N(tEB)₂-exendin-4 (52.6 h), which is defined as the time duration from 50% reduction of glucose level to the rebound to the original level, was significantly longer than the three other treatment groups (Semaglutide: 46 h, NtEB-exendin-4: 43.3 h, and exendin-4: 10.3 h) (FIG. 14d). Overall, these data demonstrated that N(tEB)₂-exendin-4 was superior to free exendin-4, and NtEB-exendin 4 in sustaining a hypoglycemic effect, with hypoglycemic potency comparable to or even greater than FDA approved semaglutide.

This disclosure further encompasses the following embodiments.

Embodiment 1: A compound of Formula I or a pharmaceutically acceptable ester, amide, solvate, or salt thereof, or a salt of such an ester or amide or a solvate of such an ester amide or salt,

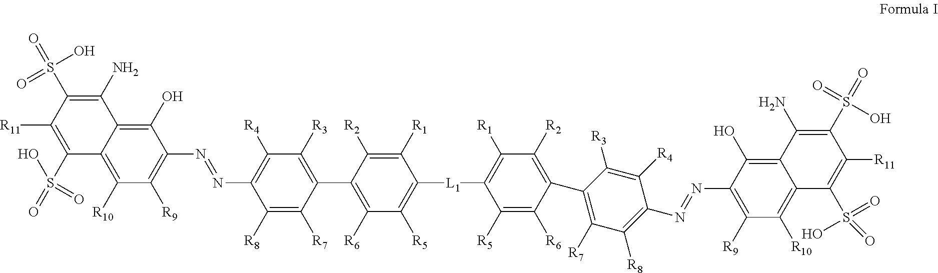

wherein:
R₁, R₂, R₃, R₄, R₅, R₆, R₇, R₈, R₉, R₁₀, and R₁₁ are chosen independently from hydrogen, halogen, hydroxyl, cyano, C₁-C₆alkyl, C₁-C₆alkoxy, C₁-C₆haloalkyl, and C₁-C₆haloalkoxy; and
L₁ is -A₁-B(A₃)-A₂-
wherein
A₁ and A₂ are chosen independently from a bond, —O—, —NH—, —NH(CO)—, —(CO)NH—, —NH(CH₂)ₘ(CO)— wherein m is an integer from 0 to 4, —(CO)(CH₂)ₖNH— wherein k is an integer from 0 to 4, —NH(CS)NH—

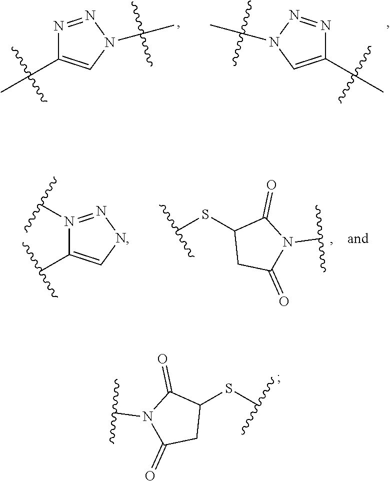

A₃ is —H, -halogen, —NH₂, —SH, —COOH, or -L₂-R₁₂, wherein
L₂ is —(CH₂)ₚ— wherein p is an integer from 0 to 12, wherein each CH₂ can be individually replaced with —O—, —S—, —NH—, —NH(CO)—, —(CO)NH—, —NH(CS)NH— provided that no two adjacent CH₂ groups are replaced;

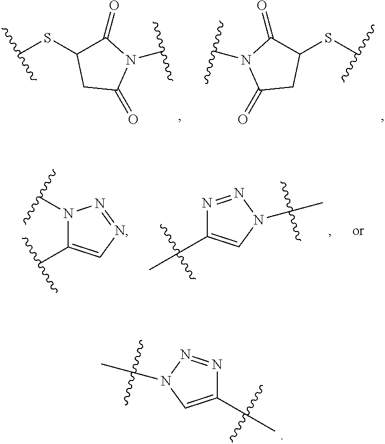 [SMILES: *SC1CC(=O)N(*)C1=O ; *SC1CC(=O)N(*)C1=O ; *c1cnnn1* ; *c1cn(*)nn1 ; *c1cn(*)nn1]

R₁₂ is —H, a chelating group, a crosslinker, or a conjugate; and
B is

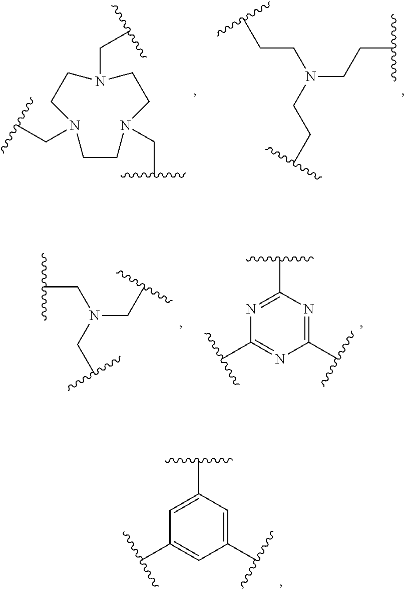 [SMILES: *CN1CCN(C*)CCN(C*)CC1 ; *CCN(CC*)CC* ; *CN(C*)C* ; *c1nc(*)nc(*)n1 ; *c1cc(*)cc(*)c1]

or —(CH₂)n- wherein n is an integer from 0 to 12, wherein each CH₂ can be individually replaced with —O—, —NH(CO)—, or —(CO)NH— providing no two adjacent CH₂ groups are replaced, and wherein —(CH₂)n- is substituted with one substituent A₃.

Embodiment 2: The compound of claim 1, wherein R₁ and R₄ are chosen independently from halogen, hydroxyl, cyano, C₁-C₆alkyl, C₁-C₆alkoxy, C₁-C₆haloalkyl, and C₁-C₆haloalkoxy.

Embodiment 3: The compound of claim 1 or 2, wherein R₁ and R₄ are chosen independently from C₁-C₆alkyl.

Embodiment 4: The compound of any one of claims 1 to 3, wherein R₁ and R₄ are each methyl, and R₂, R₃, R₅, R₆, R₇, R₈, R₉, R₁₀, and R₁₁ are each hydrogen.

Embodiment 5: The compound of any one of claims 1 to 4, wherein
A₁ is —NH(CS)NH—, A₂ is —(CO)NH—, and B is —(CH₂)₄CH(A₃)- wherein A₃ is -L₂R₁₂ and L₂ is —NH(CO)CH₂—;
A₁ is —NH(CO)—, A₂ is —(CO)NH—, and B is —(CH₂)₂CH(A₃)- wherein A₃ is —NH₂; or
A₁ is —NH(CO)—, A₂ is —(CO)NH—, and B is —CH₂CH(A₃)CH₂— wherein A₃ is —NH₂.

Embodiment 6: The compound of any one of claims 1 to 5, wherein R₁₂ is

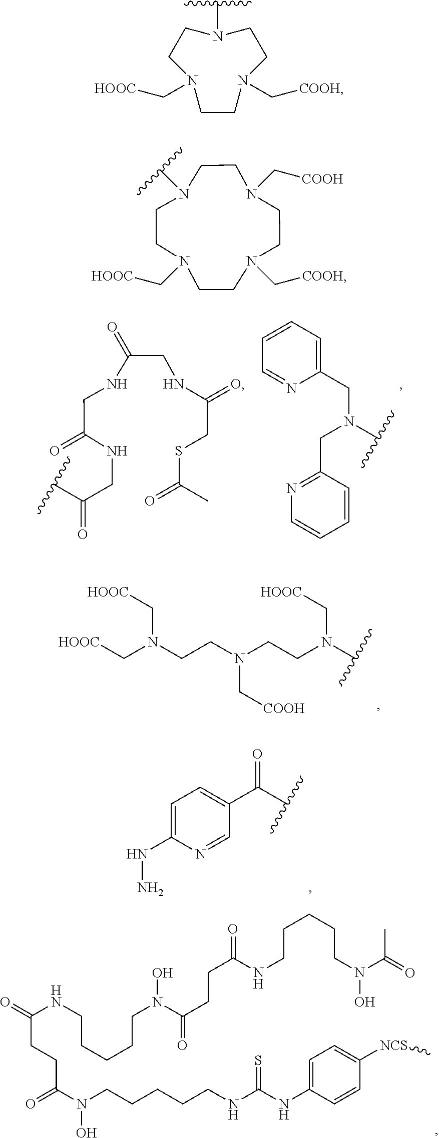

a crown ether, a cyclodextrin, or a porphyrin; preferably R₁₂ is

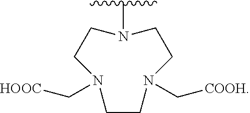 [SMILES: *N1CCN(CC(=O)O)CCN(CC(=O)O)CC1]

Embodiment 7: The compound of any one of claims 1 to 5, wherein R₁₂ is

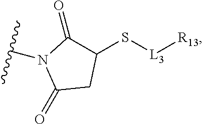

### —SH, —SR₁₃,

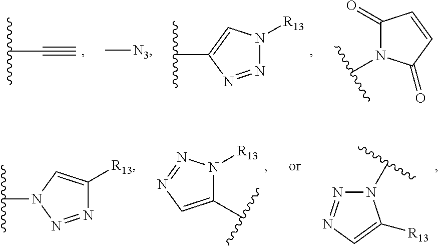

wherein
R₁₃ is hydrogen, a marker compound, a fluorescent tag, a pharmaceutically active agent, a toxin, a radioactive agent, a contrast agent, an antibody, a protein, a peptide, a peptidomimetic, a nucleic acid, a nucleic acid complex, a cytokine; preferably R₁₃ is a peptide, and
L₃ is —(CH₂)_{q}— wherein q is an integer from 0 to 12, and each CH₂ can be individually replaced with —O—, —S—, —NH(CO)—, or —(CO)—NH—, providing no two adjacent CH₂ groups are replaced.

Embodiment 8: The compound of any one of Claims 1 to 4 and 7, wherein A₁ is —NH(CH₂)ₘ(CO)— and A₂ is —(CO)(CH₂)ₖNH— wherein independently each of m and k is an integer from 0 to 4, and B is

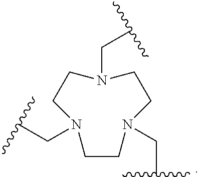 [SMILES: *CN1CCN(C*)CCN(C*)CC1]

Embodiment 9: The compound of any one of claims 1 to 4 and 7 to 8 wherein independently each of m and k is an integer from 0 to 2, preferably m=0, k=0.

Embodiment 10: The compound of any one of claims 1 to 4 and 7 to 9, wherein A₃ is —COOH.

Embodiment 11: The compound of any one of claims 1 to 4 and 7 to 10, wherein A₃ is -L₂-R₁₂, wherein L₂ is —[(CO)NH(CH₂)r]-, r is an integer from 1 to 3, and R₁₂ is

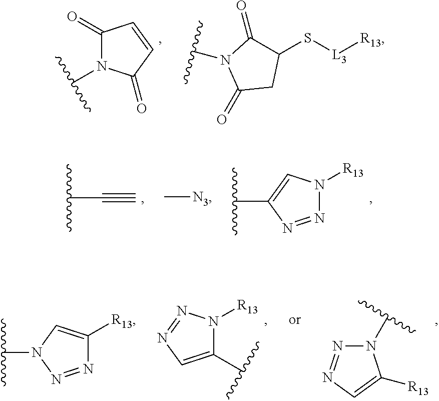

wherein
R₁₃ is a marker compound, a fluorescent tag, a pharmaceutically active agent, a toxin, a radioactive agent, a contrast agent, an antibody, a protein, a peptide, a peptidomimetic, a nucleic acid, a nucleic acid complex, or a cytokine, preferably R₁₃ is a peptide, and
L₃ is —(CH₂)_{q}— wherein q is an integer from 0 to 12, and each CH₂ can be individually replaced with —O—, —NH(CO)—, or —(CO)—NH—, providing no two adjacent CH₂ groups are replaced.

Embodiment 12: The compound of any one of Claims 1 to 4 and 7, wherein B is

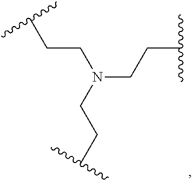 [SMILES: *CCN(CC*)CC*]

and either A₁ is —NH(CS)NH—, A₂ is —NH(CS)NH—, and A₃ is —NH₂ or -L₂-R₁₂; or A₁ is —NH(CO)—, A₂ is —(CO)NH—, and A₃ is —COOH or -L₂-R₁₂.

Embodiment 13: The compound of any one of Claims 1 to 4 and 7, wherein B is

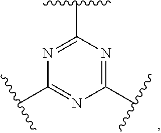 [SMILES: *c1nc(*)nc(*)n1]

### and A₁ is

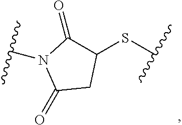 [SMILES: *SC1CC(=O)N(*)C1=O]

### A₂ is

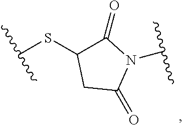 [SMILES: *SC1CC(=O)N(*)C1=O]

and A₃ is —SH or -L₂-R₁₂; or
A₁ is —NH—, A₂ is —NH—, and A₃ is —Cl or -L₂-R₁₂; or
A₁ is —NH(CO)—, A₂ is —(CO)NH—, and A₃ is —COOH or -L₂-R₁₂.

Embodiment 14: The compound of any one of claims 1 to 13, wherein the compound is one of the following:

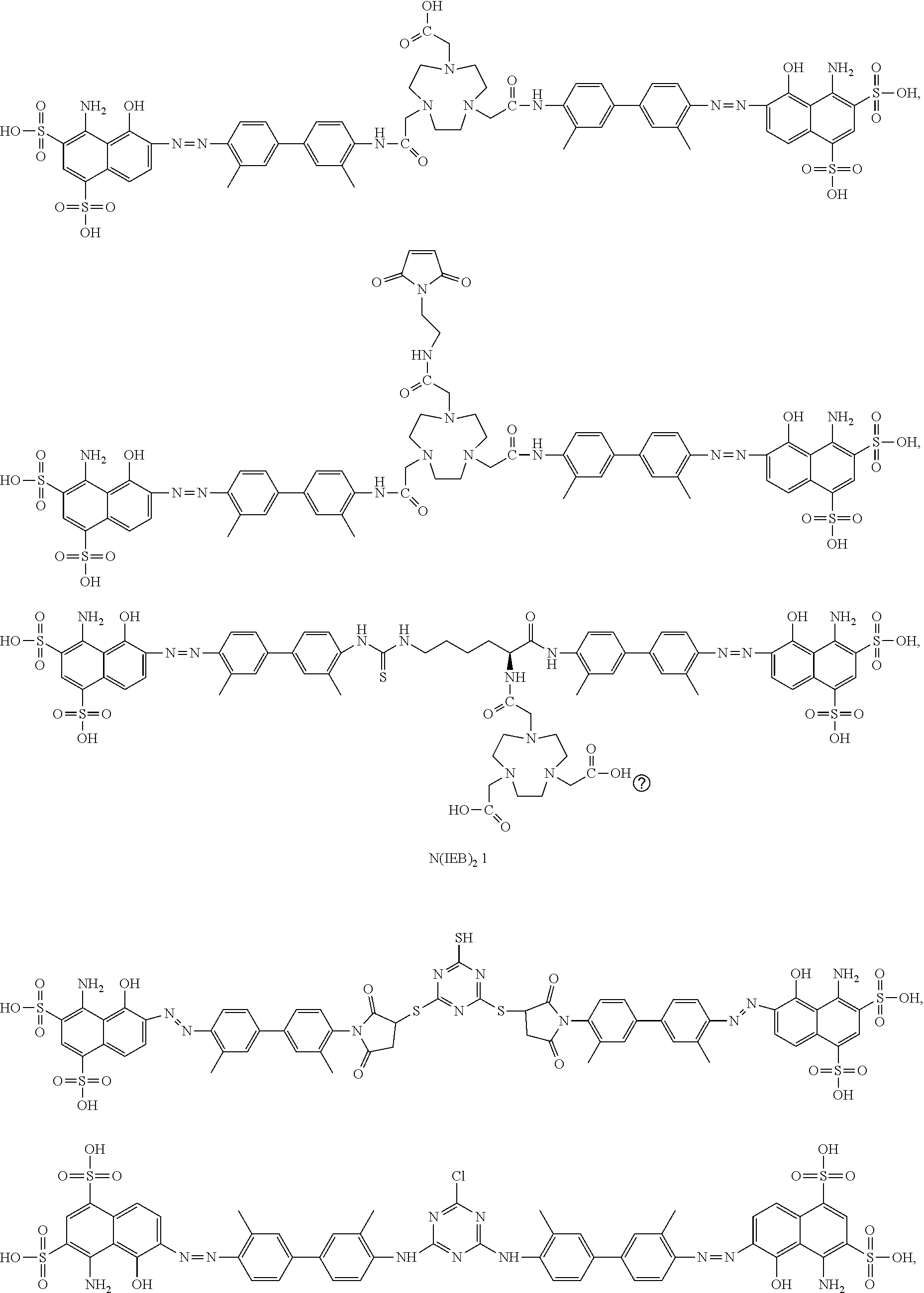

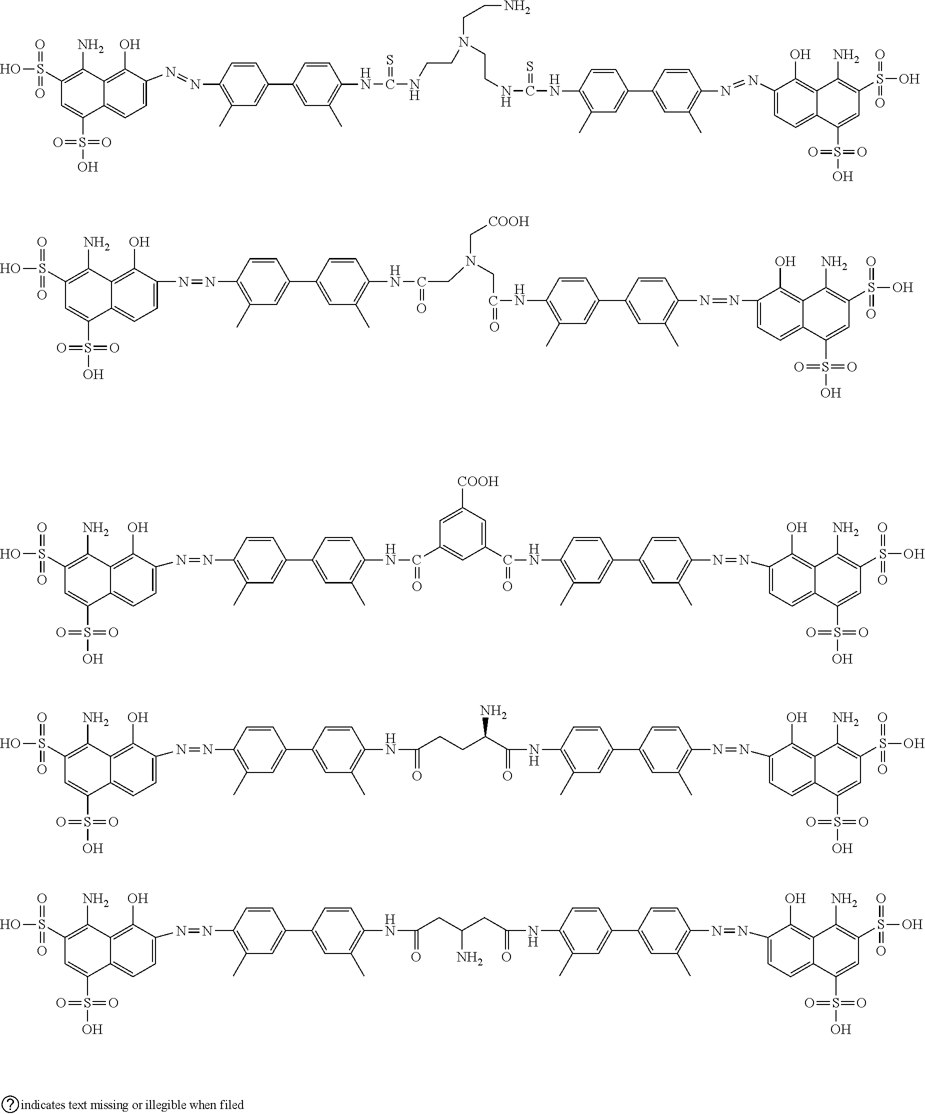

Embodiment 15: The compound of any one of claims to 1 to 14 wherein R₁₂ further comprises a radionuclide.

Embodiment 16: The compound of Claim 15, wherein the radionuclide is ¹⁸F, ⁷⁶Br, ¹²⁴I, ¹²⁵I, ¹³¹I, ⁶⁴Cu, ⁶⁷Cu, ⁹⁰Y, ⁸⁶Y, ¹¹¹In, ¹⁸⁶Re, ¹⁸⁸Re, ⁸⁹Zr, ⁹⁹Tc, ¹⁵³Sm, ²¹³Bi, ²²⁵Ac, ¹⁷⁷Lu, ²²³Ra, or a combination thereof.

Embodiment 17: The compound of any one of claims to 1 to 16, wherein R₁₃ is insulin, an insulin analog, IL-2, IL-5, GLP-1, BNP, IL-1-RA, KGF, ancestim, GH, G-CSF, CTLA-4, myostatin, Factor VII, Factor VIII, Factor IX, Exendin-4, exendin (9-39), octreotide, bombesin, RGD peptide (arginylglycylaspartic acid), vascular endothelial growth factor (VEGF), interferon (IFN), tumor necrosis factor (TNF), asparaginase, adenosine deaminase, a therapeutic fragment of any of the foregoing, a derivative of any of the foregoing, calicheamycin, auristatin, doxorubicin, maytansinoid, taxane, ecteinascidin, geldanamycin, methotrexate, camptothecin, paclitaxel, gemcitabine, temozolomide, cyclophosphamide, cyclosporine, a non-steroidal anti-inflammatory drug, a cytokine suppressive anti-inflammatory drug, a corticosteroid, methotrexate, prednisone, cyclosporine, morroniside cinnamic acid, leflunomide, or a combination thereof.

Embodiment 18: The compound of claim 17 wherein the compound is

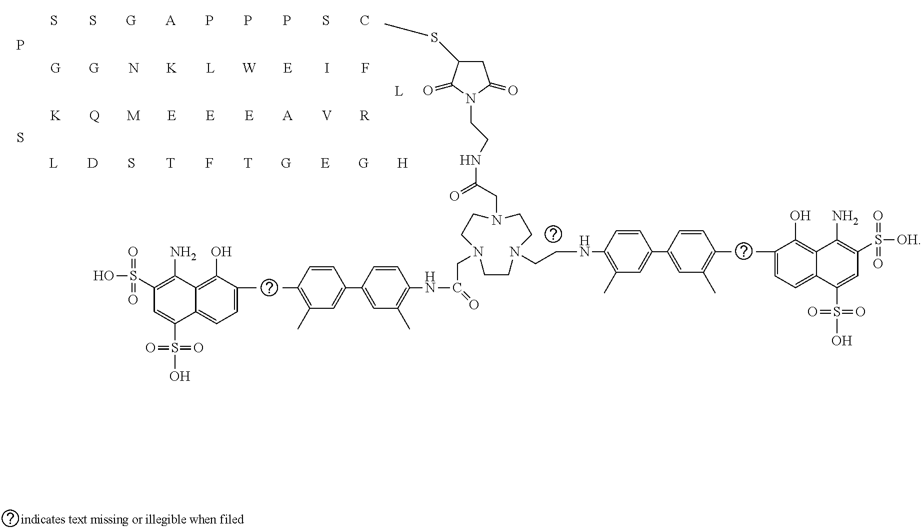

Embodiment 19: A composition comprising the compound of any one of Claims 1 to 18; and a carrier, preferably a pharmaceutically acceptable carrier.

Embodiment 20: A method of treating or diagnosing diabetes in a mammal, comprising administering to the mammal a therapeutically effective amount of the compound of any one of claims 1 to 18 or the composition of claim 19, optionally in combination with one or more additional active ingredients, preferably in the compound R₁₂ is a chelating group or a conjugate, more preferably the conjugate R₁₂ is

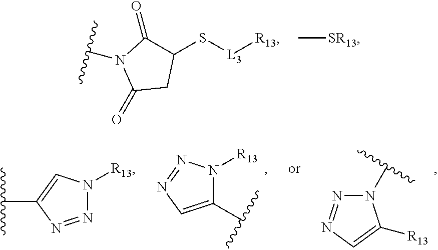

Embodiment 21: The method of claim 20, wherein the one or more additional active ingredients are selected from insulin, exenatide, dipeptidyl peptidase-4 inhibitors, neuropilin, epidermal growth factor, islet neogenesis associated protein, alpha-1 antitrypsin, anti-inflammatory agents, glulisine, glucagons, local cytokines, modulators of cytokines, anti-apoptotic molecules, aptamers, asparaginase, adenosine deaminase, interferon α2a, interferon α2b, granulocyte colony stimulating factor, growth hormone receptor antagonists, and combinations thereof.

Embodiment 22: A method of increasing the in vivo half-life of an target molecule comprising covalently coupling the compound of any one of claims 1 to 15 to a target molecule, preferably in the compound R₁₂ is a crosslinker, more preferably the crosslinker is —SH,

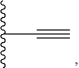 [SMILES: *C#C]

### —N₃, or

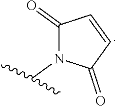 [SMILES: *N1C(=O)C=CC1=O]

Embodiment 23: The method of claim 22, wherein the target molecule is an antibody, a peptide, an anti-cancer compound, an anti-diabetes compound, or a combination thereof.

Embodiment 24: A method of in vivo imaging comprising administering to a subject a compound of any one of claims 1-18, preferably in the compound R₁₂ is a chelating group or a conjugate, more preferably the conjugate R₁₂ is

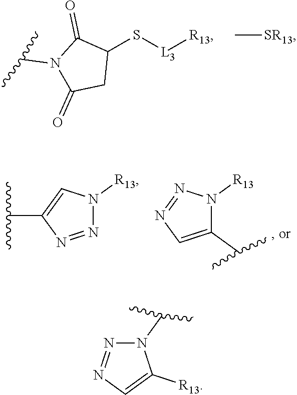

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A compound of Formula I or a pharmaceutically acceptable ester, amide, solvate, or salt thereof, or a salt of such an ester or amide or a solvate of such an ester amide or salt, 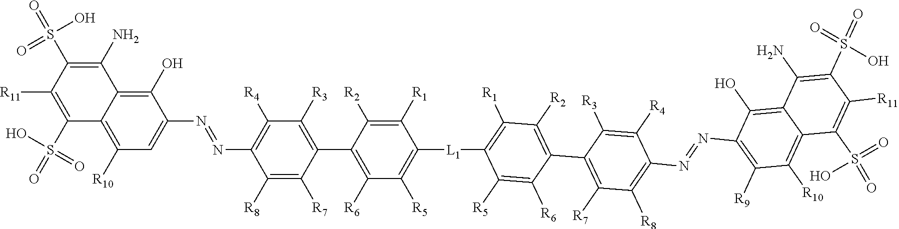
wherein:
R₁, R₂, R₃, R₄, R₅, R₆, R₇, R₈, R₉, R₁₀, and R₁₁ are chosen independently from hydrogen, halogen, hydroxyl, cyano, C₁-C₆alkyl, C₁-C₆alkoxy, C₁-C₆haloalkyl, and C₁-C₆haloalkoxy; and
L₁ is -A₁-B(A₃)-A₂-
wherein A₁ and A₂ are chosen independently from a bond, —O—, —NH—, —NH(CO)—, —(CO)NH—, —NH(CH₂)ₘ(CO)— wherein m is an integer from 0 to 4, —(CO)(CH₂)ₖNH— wherein k is an integer from 0 to 4, —NH(CS)NH—, 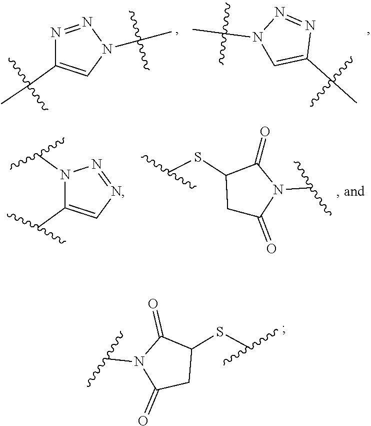 [SMILES: *c1cn(*)nn1 ; *c1cn(*)nn1 ; *c1cnnn1* ; *SC1CC(=O)N(*)C1=O ; *SC1CC(=O)N(*)C1=O]
A₃ is —H, -halogen, —NH₂, —SH, —COOH, or -L₂-R₁₂, wherein
L₂ is —(CH₂)ₚ— wherein p is an integer from 0 to 12, wherein each CH₂ can be individually replaced with —O—, —S—, —NH—, —NH(CO)—, —(CO)NH—, —NH(CS)NH— provided that no two adjacent CH₂ groups are replaced; 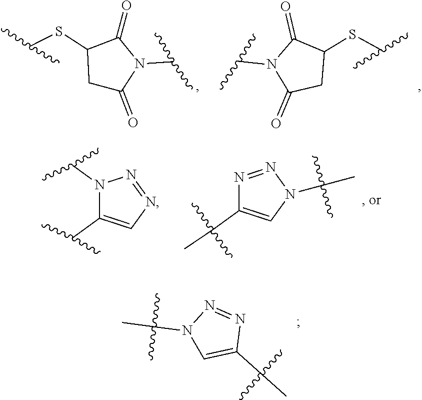
R₁₂ is —H, a chelating group, a crosslinker, or a conjugate; and
B is 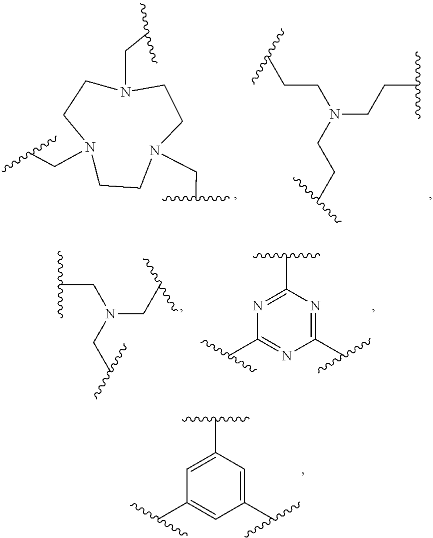 [SMILES: *CN1CCN(C*)CCN(C*)CC1 ; *CCN(CC*)CC* ; *CN(C*)C* ; *c1nc(*)nc(*)n1 ; *c1cc(*)cc(*)c1]
or
—(CH₂)n- wherein n is an integer from 0 to 12, wherein each CH₂ can be individually replaced with —O—, —NH(CO)—, or —(CO)NH— providing no two adjacent CH₂ groups are replaced, and wherein —(CH₂)n- is substituted with one substituent A₃.

2. The compound of claim 1, wherein R₁ and R₄ are chosen independently from halogen, hydroxyl, cyano, C₁-C₆alkyl, C₁-C₆alkoxy, C₁-C₆haloalkyl, and C₁-C₆haloalkoxy.

3. (canceled)

4. The compound of claim 1, wherein R₁ and R₄ are each methyl, and R₂, R₃, R₅, R₆, R₇, R₈, R₉, R₁₀, and Ru are each hydrogen.

5. The compound of claim 1, wherein
A₁ is —NH(CS)NH—, A₂ is —(CO)NH—, and B is —(CH₂)₄CH(A₃)- wherein A₃ is (-L₂R₁₂) and
L₂ is —NH(CO)CH₂—;
A₁ is —NH(CO)—, A₂ is —(CO)NH—, and B is —(CH₂)₂CH(A₃)- wherein A₃ is —NH₂; or
A₁ is —NH(CO)—, A₂ is —(CO)NH—, and B is —CH₂CH(A₃)CH₂— wherein A₃ is —NH₂.

6. The compound of claim 1, 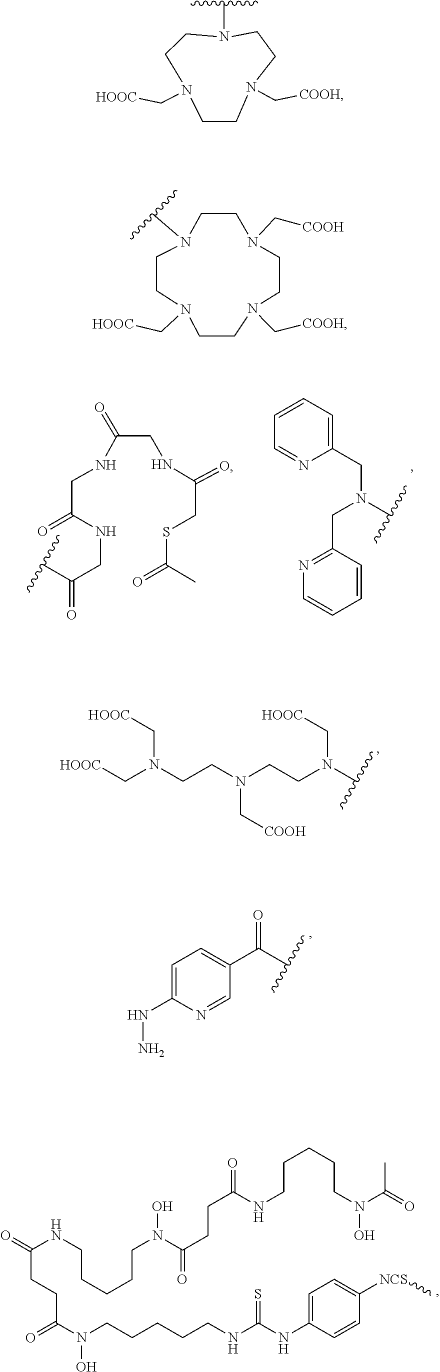
a crown ether, a cyclodextrin, or a porphyrin.

7. The compound of claim 1, wherein R₁₂ is 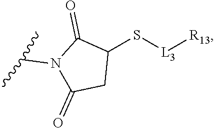
—SH, —SR₁₃, 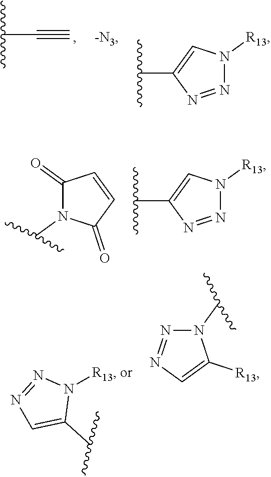
wherein R₁₃ is hydrogen, a marker compound, a fluorescent tag, a pharmaceutically active agent, a toxin, a radioactive agent, a contrast agent, an antibody, a protein, a peptide, a peptidomimetic, a nucleic acid, a nucleic acid complex, a cytokine;
L₃ is —(CH₂)_{q}— wherein q is an integer from 0 to 12, and each CH₂ can be individually replaced with —O—, —S—, —NH(CO)—, or —(CO)—NH—, providing no two adjacent CH₂ groups are replaced.

8. The compound of claim 1, wherein A₁ is —NH(CH₂)ₘ(CO)— and A₂ is —(CO)(CH₂)ₖNH— wherein independently each of m and k is an integer from 0 to 4, and B is

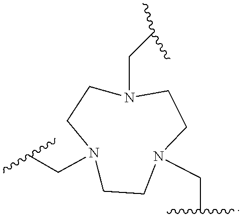 [SMILES: *CN1CCN(C*)CCN(C*)CC1]

9. The compound of claim 1 wherein independently each of m and k is an integer from 0 to 2, preferably m=0, k=0.

10. The compound of claim 1, wherein A₃ is —COOH.

11. The compound of claim 1, wherein A₃ is -L₂-R₁₂, wherein L₂ is —[(CO)NH(CH₂)r]-, r is an integer from 1 to 3, and R₁₂ is 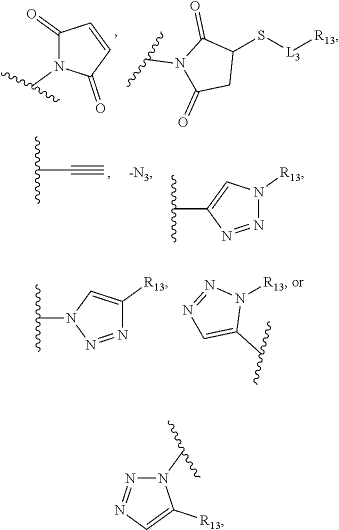
wherein
R₁₃ is a marker compound, a fluorescent tag, a pharmaceutically active agent, a toxin, a radioactive agent, a contrast agent, an antibody, a protein, a peptide, a peptidomimetic, a nucleic acid, a nucleic acid complex, or a cytokine, and
L₃ is —(CH₂)_{q}— wherein q is an integer from 0 to 12, and each CH₂ can be individually replaced with —O—, —NH(CO)—, or —(CO)—NH—, providing no two adjacent CH₂ groups are replaced.

12. The compound of claim 1, wherein B is 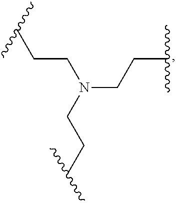 [SMILES: *CCN(CC*)CC*]
and
A₁ is —NH(CS)NH—, A₂ is —NH(CS)NH—, and A₃ is —NH₂ or -L₂-R₁₂; or
A₁ is —NH(CO)—, A₂ is —(CO)NH—, and A₃ is —COOH or -L₂-R₁₂.

13. The compound of claim 1, wherein B is 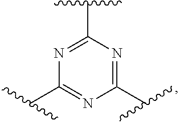 [SMILES: *c1nc(*)nc(*)n1]
and
A₁ is 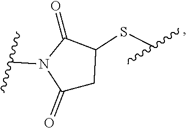 [SMILES: *SC1CC(=O)N(*)C1=O]
A₂ is 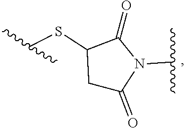 [SMILES: *SC1CC(=O)N(*)C1=O]
and A₃ is —SH or -L₂-R₁₂; or
A₁ is —NH—, A₂ is —NH—, and A₃ is —Cl or -L₂-R₁₂; or
A₁ is —NH(CO)—, A₂ is —(CO)NH—, and A₃ is —COOH or -L₂-R₁₂.

14. The compound of claim 1, wherein the compound is one of the following:

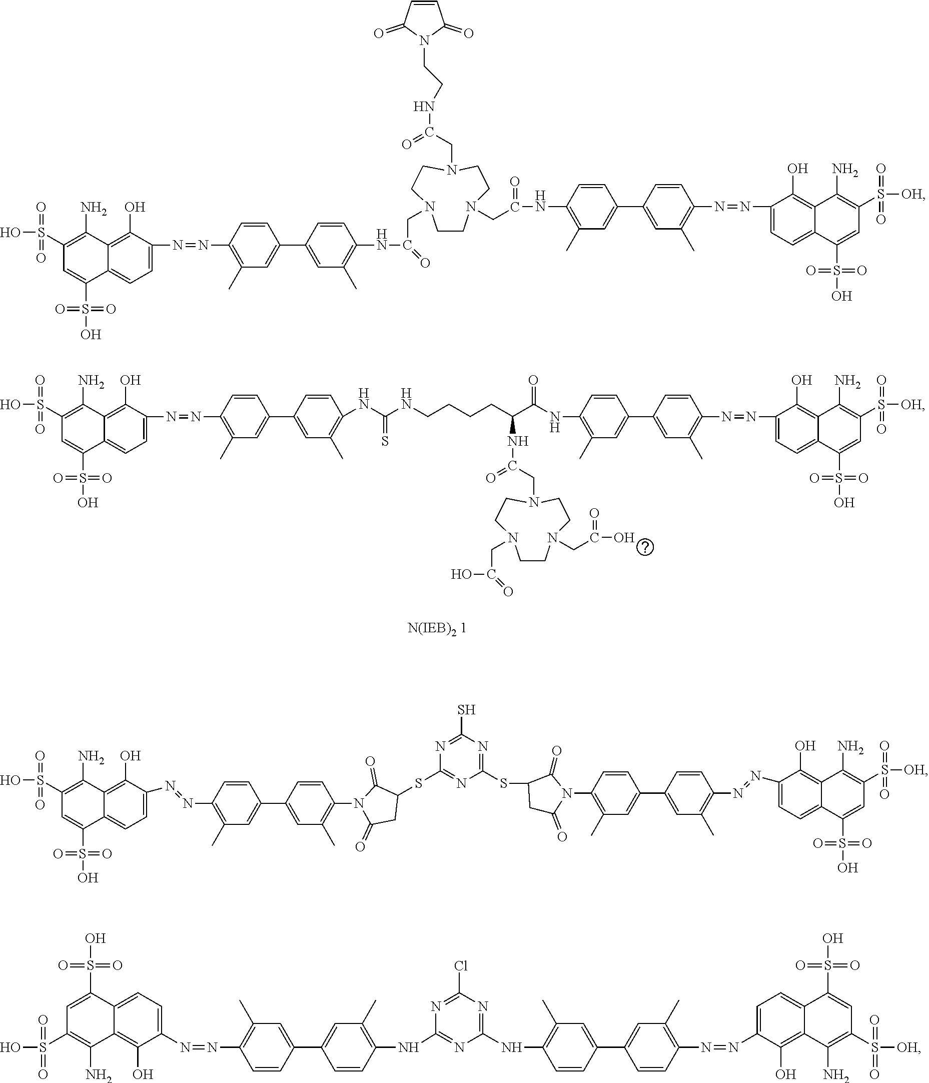

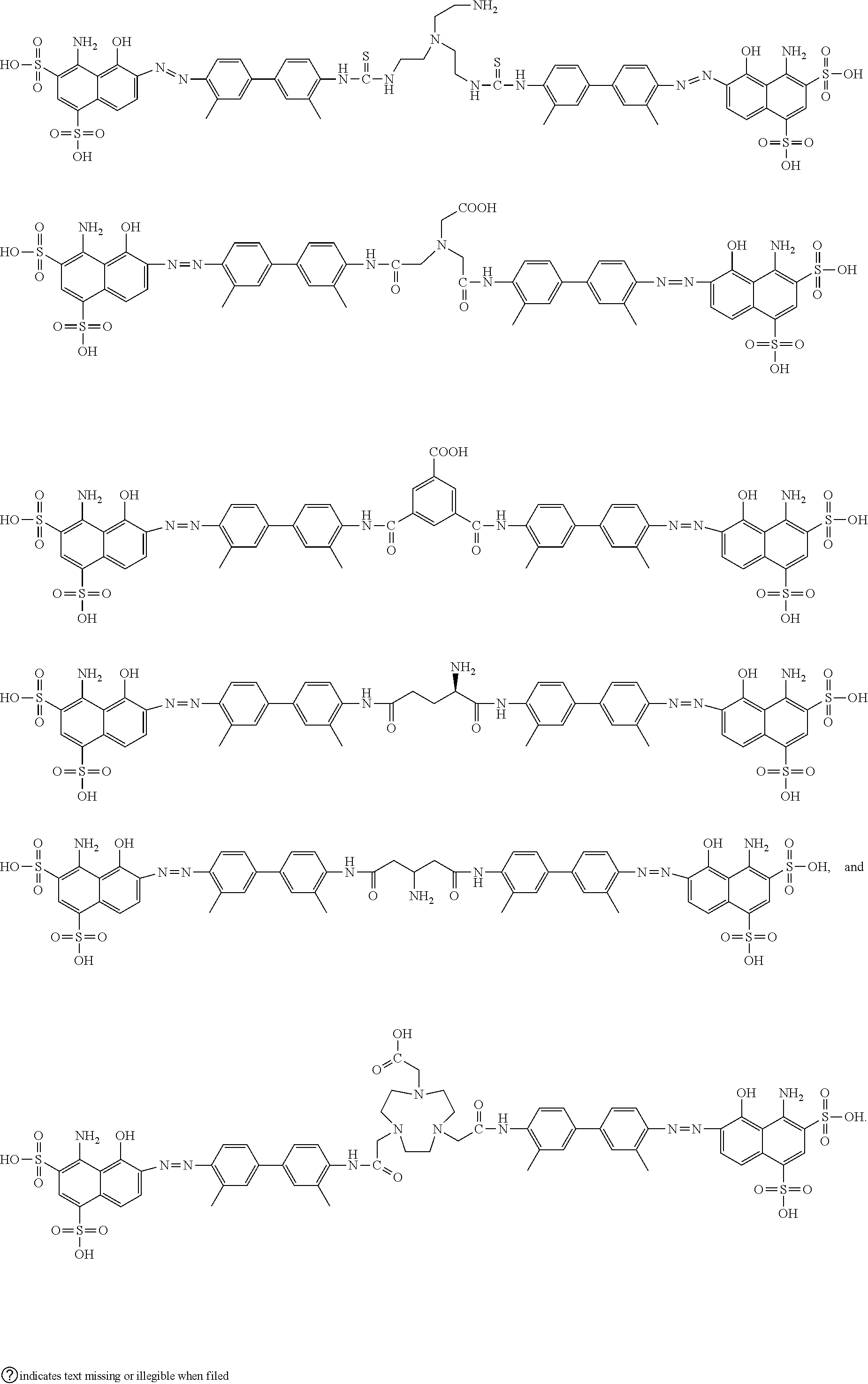

15. The compound of claim 1
wherein R₁₂ further comprises a radionuclide.

16. The compound of claim 15, wherein the radionuclide is ¹⁸F, ⁷⁶Br, ¹²⁴I, ¹²⁵I, ¹³¹a, ⁶⁴Cu, ⁶⁷Cu, ⁹⁰Y, ⁸⁶Y, ¹¹¹In, ¹⁸⁶Re, ¹⁸⁸Re, ⁸⁹Zr, ⁹⁹Tc, ¹⁵³Sm, ²¹³Bi, ²²⁵Ac, ¹⁷⁷Lu, ²²³Ra, or a combination thereof.

17. The compound of claim 1, wherein R₁₃ is insulin, an insulin analog, IL-2, IL-5, GLP-1, BNP, IL-1-RA, KGF, ancestim, GH, G-CSF, CTLA-4, myostatin, Factor VII, Factor VIII, Factor IX, Exendin-4, exendin (9-39), octreotide, bombesin, RGD peptide (arginylglycylaspartic acid), vascular endothelial growth factor (VEGF), interferon (IFN), tumor necrosis factor (TNF), asparaginase, adenosine deaminase, a therapeutic fragment of any of the foregoing, a derivative of any of the foregoing, calicheamycin, auristatin, doxorubicin, maytansinoid, taxane, ecteinascidin, geldanamycin, methotrexate, camptothecin, paclitaxel, gemcitabine, temozolomide, cyclophosphamide, cyclosporine, a non-steroidal anti-inflammatory drug, a cytokine suppressive anti-inflammatory drug, a corticosteroid, methotrexate, prednisone, cyclosporine, morroniside cinnamic acid, leflunomide, or a combination thereof.

18. The compound of claim 17 wherein the compound is

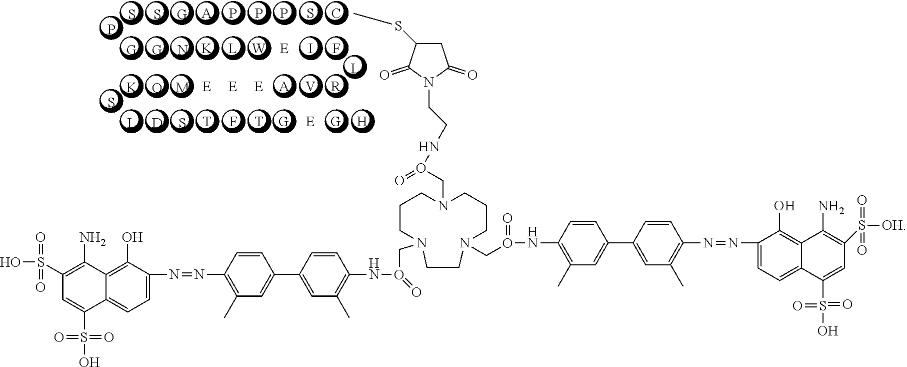

19. A composition comprising the compound of claim 1; and a carrier, preferably a pharmaceutically acceptable carrier.

20. A method of treating or diagnosing diabetes in a mammal, comprising administering to the mammal a therapeutically effective amount of the compound of claim 1, optionally in combination with one or more additional active ingredients, preferably in the compound R₁₂ is a chelating group or a conjugate.

21. (canceled)

22. A method of increasing the in vivo half-life of an target molecule comprising covalently coupling the compound of claim 1 to a target molecule, preferably in the compound R₁₂ is a crosslinker.

23. The method of claim 22, wherein the target molecule is an antibody, a peptide, an anti-cancer compound, an anti-diabetes compound, or a combination thereof.

24. A method of in vivo imaging comprising administering to a subject a compound of claim 1.


SEQUENCE LISTING (1 sequence)

SEQ ID NO: 1 - protein, 40 aa, Artificial Sequence
  Cys-40 Exendin-4
HGEGTFTSDLSKQMEEEAVRLFIEWLKNGGPSSGAPPPSC